(19) United States
(12) Patent Application Publication
(10) Pub. No.: US 2023/0035074 A1
(43) Pub. Date: Feb. 2, 2023

(54) Intrasacular Aneurysm Occlusion Device with Nested Structures

(71) Applicant: Robert A. Connor, St. Paul, MN (US)

(72) Inventor: Robert A. Connor, St. Paul, MN (US)

(73) Assignee: Aneuclose LLC, St. Paul, MN (US)

(21) Appl. No.: 17/965,502

(22) Filed: Oct. 13, 2022

Related U.S. Application Data
(63) Continuation-in-part of application No. 17/829,313, filed on May 31, 2022.
Application No. 17/829,313 is a continuation-in-part of application No. 17/476,845, filed on Sep. 16, 2021.
Application No. 17/829,313 is a continuation-in-part of application No. 17/485,390, filed on Sep. 25, 2021, now Pat. No. 11,471,164.
Application No. 17/485,390 is a continuation-in-part of application No. 17/476,845, filed on Sep. 16, 2021.
Application No. 17/476,845 is a continuation-in-part of application No. 17/472,674, filed on Sep. 12, 2021, now abandoned.
Application No. 17/472,674 is a continuation-in-part of application No. 17/467,680, filed on Sep. 7, 2021, now abandoned.
Application No. 17/467,680 is a continuation-in-part of application No. 17/466,497, filed on Sep. 3, 2021, now Pat. No. 11,357,511.
Application No. 17/466,497 is a continuation-in-part of application No. 17/353,652, filed on Jun. 21, 2021, now abandoned.
Application No. 17/353,652 is a continuation-in-part of application No. 17/220,002, filed on Apr. 1, 2021, now Pat. No. 11,464,518.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/220,002 is a continuation-in-part of application No. 17/214,827, filed on Mar. 27, 2021, now Pat. No. 11,484,322.
Application No. 17/214,827 is a continuation-in-part of application No. 17/211,446, filed on Mar. 24, 2021, now abandoned.
Application No. 17/211,446 is a continuation-in-part of application No. 16/693,267, filed on Nov. 23, 2019, now abandoned.
Application No. 16/693,267 is a continuation-in-part of application No. 16/660,929, filed on Oct. 23, 2019, now Pat. No. 11,471,163.
Application No. 16/693,267 is a continuation-in-part of application No. 16/660,929, filed on Oct. 23, 2019, now Pat. No. 11,471,163.
Application No. 16/660,929 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 16/541,241, filed on Aug. 15, 2019, now abandoned.
Application No. 16/541,241 is a continuation-in-part of application No. 15/865,822, filed on Jan. 9, 2018, now Pat. No. 10,716,573.
Application No. 15/865,822 is a continuation-in-part of application No. 15/861,482, filed on Jan. 3, 2018, now abandoned.
Application No. 15/861,482 is a continuation-in-part of application No. 15/865,822, filed on Jan. 9, 2018, now Pat. No. 10,716,573.
Application No. 15/865,822 is a continuation-in-part of application No. 15/081,909, filed on Mar. 27, 2016, now abandoned.
Application No. 15/081,909 is a continuation-in-part of application No. 14/526,600, filed on Oct. 29, 2014, now abandoned.
Application No. 14/526,600 is a continuation-in-part of application No. 15/080,915, filed on Mar. 25, 2016, now Pat. No. 10,028,747.
Application No. 15/080,915 is a continuation-in-part of application No. 14/526,600, filed on Oct. 29, 2014, now abandoned.
Application No. 15/081,909 is a continuation-in-part of application No. 14/526,600, filed on Oct. 29, 2014, now abandoned.
Application No. 15/080,915 is a continuation-in-part of application No. 14/526,600, filed on Oct. 29, 2014, now abandoned.
Application No. 14/526,600 is a continuation-in-part of application No. 12/989,048, filed on Oct. 21, 2010, now Pat. No. 8,974,487.
(60) Provisional application No. 63/119,774, filed on Dec. 1, 2020.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/794,609, filed on Jan. 19, 2019.
(60) Provisional application No. 62/794,607, filed on Jan. 19, 2019.
(60) Provisional application No. 62/720,173, filed on Aug. 21, 2018.
(60) Provisional application No. 62/589,754, filed on Nov. 22, 2017.
(60) Provisional application No. 62/472,519, filed on Mar. 16, 2017.
(60) Provisional application No. 62/589,754, filed on Nov. 22, 2017.
(60) Provisional application No. 62/472,519, filed on Mar. 16, 2017.
(60) Provisional application No. 62/444,860, filed on Jan. 11, 2017.
(60) Provisional application No. 61/897,245, filed on Oct. 30, 2013.

(51) Int. Cl. A61B 17/12

(52) U.S. Cl. CPC A61B 17/12113; A61B 17/12172; A61B 17/12177; A61B 2017/1205; A61B 17/12181

(57) ABSTRACT

This invention is an intrasacular aneurysm occlusion device with nested structures, including an inner embolic structure (e.g. inner mesh, net, braid, and/or stent) which is nested inside an outer embolic structure (e.g. outer mesh, net, braid, and/or stent). The inner embolic structure can have an ellipsoidal, toroidal, inverted jug, or inverted bottle shape. The outer embolic structure can have a spherical shape. The device can also have an opening and valve through which embolic material is inserted into the aneurysm sac.

### CROSS-REFERENCE TO RELATED APPLICATIONS

This application is a continuation-in-part of U.S. patent application Ser. No. 17/829,313 filed on 2022 May 31 and is a continuation-in-part of U.S. patent application Ser. No. 17/476,845 filed on 2021 Sep. 16.

U.S. patent application Ser. No. 17/829,313 was a continuation-in-part of U.S. patent application Ser. No. 17/485,390 filed on 2021 Sep. 25, was a continuation-in-part of U.S. patent application Ser. No. 17/476,845 filed on 2021 Sep. 16, was a continuation-in-part of U.S. patent application Ser. No. 17/472,674 filed on 2021 Sep. 12, was a continuation-in-part of U.S. patent application Ser. No. 17/467,680 filed on 2021 Sep. 7, was a continuation-in-part of U.S. patent application Ser. No. 17/466,497 filed on 2021 Sep. 3, was a continuation-in-part of U.S. patent application Ser. No. 17/353,652 filed on 2021 Jun. 21, was a continuation-in-part of U.S. patent application Ser. No. 17/220,002 filed on 2021 Apr. 1, was a continuation-in-part of U.S. patent application Ser. No. 17/214,827 filed on 2021 Mar. 27, was a continuation-in-part of U.S. patent application Ser. No. 17/211,446 filed on 2021 Mar. 24, was a continuation-in-part of U.S. patent application Ser. No. 16/693,267 filed on 2019 Nov. 23, and was a continuation-in-part of U.S. patent application Ser. No. 16/660,929 filed on 2019 Oct. 23.

U.S. patent application Ser. No. 17/220,002 was a continuation-in-part of U.S. patent application Ser. No. 17/214,827 filed on 2021 Mar. 27. U.S. patent application Ser. No. 17/220,002 was a continuation-in-part of U.S. patent application Ser. No. 17/211,446 filed on 2021 Mar. 24. U.S. patent application Ser. No. 17/220,002 claimed the priority benefit of U.S. provisional patent application 63/119,774 filed on 2020 Dec. 1. U.S. patent application Ser. No. 17/220,002 was a continuation-in-part of U.S. patent application Ser. No. 16/693,267 filed on 2019 Nov. 23. U.S. patent application Ser. No. 17/220,002 was a continuation-in-part of U.S. patent application Ser. No. 16/660,929 filed on 2019 Oct. 23.

U.S. patent application Ser. No. 16/693,267 was a continuation-in-part of U.S. patent application Ser. No. 16/660,929 filed on 2019 Oct. 23. U.S. patent application Ser. No. 16/693,267 claimed the priority benefit of U.S. provisional patent application 62/794,609 filed on 2019 Jan. 19. U.S. patent application Ser. No. 16/693,267 claimed the priority benefit of U.S. provisional patent application 62/794,607 filed on 2019 Jan. 19. U.S. patent application Ser. No. 16/693,267 was a continuation-in-part of U.S. patent application Ser. No. 16/541,241 filed on 2019 Aug. 15. U.S. patent application Ser. No. 16/693,267 was a continuation-in-part of U.S. patent application Ser. No. 15/865,822 filed on 2018 Jan. 9 which issued as U.S. patent Ser. No. 10/716,573 on 2020 Jul. 21. U.S. patent application Ser. No. 16/693,267 was a continuation-in-part of U.S. patent application Ser. No. 15/861,482 filed on 2018 Jan. 3.

U.S. patent application Ser. No. 16/660,929 claimed the priority benefit of U.S. provisional patent application 62/794,609 filed on 2019 Jan. 19. U.S. patent application Ser. No. 16/660,929 claimed the priority benefit of U.S. provisional patent application 62/794,607 filed on 2019 Jan. 19. U.S. patent application Ser. No. 16/660,929 was a continuation-in-part of U.S. patent application Ser. No. 16/541,241 filed on 2019 Aug. 15. U.S. patent application Ser. No. 16/660,929 was a continuation-in-part of U.S. patent application Ser. No. 15/865,822 filed on 2018 Jan. 9 which issued as U.S. patent Ser. No. 10/716,573 on 2020 Jul. 21. U.S. patent application Ser. No. 16/660,929 was a continuation-in-part of U.S. patent application Ser. No. 15/861,482 filed on 2018 Jan. 3.

U.S. patent application Ser. No. 16/541,241 claimed the priority benefit of U.S. provisional patent application 62/794,609 filed on 2019 Jan. 19. U.S. patent application Ser. No. 16/541,241 claimed the priority benefit of U.S. provisional patent application 62/794,607 filed on 2019 Jan. 19. U.S. patent application Ser. No. 16/541,241 claimed the priority benefit of U.S. provisional patent application 62/720,173 filed on 2018 Aug. 21. U.S. patent application Ser. No. 16/541,241 was a continuation-in-part of U.S. patent application Ser. No. 15/865,822 filed on 2018 Jan. 9 which issued as U.S. patent Ser. No. 10/716,573 on 2020 Jul. 21

U.S. patent application Ser. No. 15/865,822 claimed the priority benefit of U.S. provisional patent application 62/589,754 filed on 2017 Nov. 22. U.S. patent application Ser. No. 15/865,822 claimed the priority benefit of U.S. provisional patent application 62/472,519 filed on 2017 Mar. 16. U.S. patent application Ser. No. 15/865,822 was a continuation-in-part of U.S. patent application Ser. No. 15/081,909 filed on 2016 Mar. 27. U.S. patent application Ser. No. 15/865,822 was a continuation-in-part of U.S. patent application Ser. No. 14/526,600 filed on 2014 Oct. 29.

U.S. patent application Ser. No. 15/861,482 claimed the priority benefit of U.S. provisional patent application 62/589,754 filed on 2017 Nov. 22. U.S. patent application Ser. No. 15/861,482 claimed the priority benefit of U.S. provisional patent application 62/472,519 filed on 2017 Mar. 16. U.S. patent application Ser. No. 15/861,482 claimed the priority benefit of U.S. provisional patent application 62/444,860 filed on 2017 Jan. 11. U.S. patent application Ser. No. 15/861,482 was a continuation-in-part of U.S. patent application Ser. No. 15/080,915 filed on 2016 Mar. 25 which issued as U.S. patent Ser. No. 10/028,747 on 2018 Jul. 24. U.S. patent application Ser. No. 15/861,482 was a continuation-in-part of U.S. patent application Ser. No. 14/526,600 filed on 2014 Oct. 29.

U.S. patent application Ser. No. 15/081,909 was a continuation-in-part of U.S. patent application Ser. No. 14/526,600 filed on 2014 Oct. 29. U.S. patent application Ser. No. 15/080,915 was a continuation-in-part of U.S. patent application Ser. No. 14/526,600 filed on 2014 Oct. 29. U.S. patent application Ser. No. 14/526,600 claimed the priority benefit of U.S. provisional patent application 61/897,245 filed on 2013 Oct. 30. U.S. patent application Ser. No. 14/526,600 was a continuation-in-part of U.S. patent application Ser. No. 12/989,048 filed on 2010 Oct. 21 which issued as U.S. Pat. No. 8,974,487 on 2015 Mar. 10. U.S. patent application Ser. No. 12/989,048 claimed the priority benefit of U.S. provisional patent application 61/126,047 filed on 2008 May 1. U.S. patent application Ser. No. 12/989,048 claimed the priority benefit of U.S. provisional patent application 61/126,027 filed on 2008 May 1.

The entire contents of these related applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to devices and methods for occluding a cerebral aneurysm.

### INTRODUCTION

An aneurysm is an abnormal bulging of a blood vessel wall. The vessel from which the aneurysm protrudes is the parent vessel. Saccular aneurysms look like a sac protruding out from the parent vessel. Saccular aneurysms have a neck and can be prone to rupture. Fusiform aneurysms are a form of aneurysm in which a blood vessel is expanded circumferentially in all directions. Fusiform aneurysms generally do not have a neck and are less prone to rupturing than saccular aneurysms. As an aneurysm grows larger, its walls generally become thinner and weaker. This decrease in wall integrity, particularly for saccular aneurysms, increases the risk of the aneurysm rupturing and hemorrhaging blood into the surrounding tissue, with serious and potentially fatal health outcomes.

Cerebral aneurysms, also called brain aneurysms or intracranial aneurysms, are aneurysms that occur in the intercerebral arteries that supply blood to the brain. The majority of cerebral aneurysms form at the junction of arteries at the base of the brain that is known as the Circle of Willis where arteries come together and from which these arteries send branches to different areas of the brain. Although identification of intact aneurysms is increasing due to increased use of outpatient imaging such as outpatient MRI scanning, many cerebral aneurysms still remain undetected unless they rupture. If they do rupture, they often cause stroke, disability, and/or death. The prevalence of cerebral aneurysms is generally estimated to be in the range of 1%-5% of the general population or approximately 3-15 million people in the U.S. alone. Approximately 30,000 people per year suffer a ruptured cerebral aneurysm in the U.S. alone. Approximately one-third to one-half of people who suffer a ruptured cerebral aneurysm die within one month of the rupture. Sadly, even among those who survive, approximately one-half suffer significant and permanent deterioration of brain function. Better alternatives for cerebral aneurysm treatment are needed.

### REVIEW OF THE PRIOR ART

There has been considerable innovation concerning intrasacular devices to occlude cerebral aneurysms, including the following relevant art. U.S. patent application 20220031334 (Aguilar, Feb. 3, 2022, “Expandable Devices for Treating Body Lumens”) discloses an occlusive device comprising an expandable mesh including an outer mesh and an inner mesh disposed within the outer mesh. U.S. patent applications 20170079661 (Bardsley et al., Mar. 23, 2017, “Occlusive Devices”) and 20190269411 (Bardsley et al., Sep. 5, 2019, “Occlusive Devices”) and U.S. patent Ser. No. 10/314,593 (Bardsley et al., Jun. 11, 2019, “Occlusive Devices”) disclose an implant with a single- or dual-layer braided body with variable porosity. U.S. patent application 20120283768 (Cox et al., Nov. 8, 2012, “Method and Apparatus for the Treatment of Large and Giant Vascular Defects”) discloses deployment of multiple permeable shell devices within a single vascular defect.

U.S. patent application 20170281194 (Divino et al., Oct. 5, 2017, “Embolic Medical Devices”) discloses an occlusive device with a collapsed configuration in which first and second side edges are curled toward each other. U.S. patent application 20210275187 (Franano et al., Sep. 9, 2021, “Expandable Body Device and Method of Use”) discloses medical devices comprising a single-lobed, thin-walled, expandable body.

U.S. patent application 20190365385 (Gorochow et al., Dec. 5, 2019, “Aneurysm Device and Delivery System”) and U.S. patent Ser. No. 10/939,915 (Gorochow et al., Mar. 9, 2021, “Aneurysm Device and Delivery System”) disclose a braid, wherein translating the braid causes a delivery portion to expand and form a distal sack as well as invert into itself. U.S. patent applications 20210085333 (Gorochow et al., Mar. 25, 2021, “Inverting Braided Aneurysm Treatment System and Method”), 20210186518 (Gorochow et al., Jun. 24, 2021, “Implant Having an Intrasaccular Section and Intravascular Section”), 20220104829 (Gorochow et al., Apr. 7, 2022, “Inverting Braided Aneurysm Treatment System and Method”) and U.S. patent Ser. No. 11/278,292 (Gorochow et al., Mar. 22, 2022, “Inverting Braided Aneurysm Treatment System and Method”) and U.S. Pat. No. 11,457,926 (Gorochow et al., Oct. 4, 2022, “Implant Having an Intrasaccular Section and Intravascular Section”) disclose a tubular braid with an intrasaccular section, an intravascular section, a pinched section, and a predetermined shape.

U.S. patent application 20220202425 (Gorochow et al., Jun. 30, 2022, “Semispherical Braided Aneurysm Treatment System and Method”) discloses a tubular braid with three segments and two inversions, one of the three segments extending between the two inversions and forming a sack. U.S. patent Ser. No. 11/058,430 (Gorochow et al., Jul. 13, 2021, “Aneurysm Device and Delivery System”) discloses a braid with a proximal expandable portion for positioning inside an aneurysm and sealing across the neck of the aneurysm. U.S. patent application 20210338247 (Gorochow, Nov. 4, 2021, “Double Layer Braid”) discloses a double layered braid for treating an aneurysm.

U.S. patent application 20220257260 (Hewitt et al, Aug. 18, 2022, “Filamentary Devices for Treatment of Vascular Defects”) discloses an implant having multiple mesh layers. U.S. patent applications 20160367260 (Hewitt et al., Dec. 22, 2016, “Devices for Therapeutic Vascular Procedures”) and 20170128077 (Hewitt et al., May 11, 2017, “Devices for Therapeutic Vascular Procedures”) and U.S. Pat. No. 9,629,635 (Hewitt et al., Apr. 25, 2017, “Devices for Therapeutic Vascular Procedures”) disclose a self-expanding resilient permeable shell and a metallic coil secured to the distal end of the permeable shell. U.S. patent applications 20140358178 (Hewitt et al., Dec. 4, 2014, “Filamentary Devices for Treatment of Vascular Defects”), 20160249934 (Hewitt et al., Sep. 1, 2016, “Filamentary Devices for Treatment of Vascular Defects”), 20180206849 (Hewitt et al., Jul. 26, 2018, “Filamentary Devices for the Treatment of Vascular Defects”), 20210007754 (Milhous et al., Jan. 14, 2021, “Filamentary Devices for Treatment of Vascular Defects”), and 20210275184 (Hewitt et al., Sep. 9, 2021, “Filamentary Devices for Treatment of Vascular Defects”) and U.S. Pat. No. 9,078,658 (Hewitt et al., Jul. 14, 2015, “Filamentary Devices for Treatment of Vascular Defects”), U.S. Pat. No. 9,955,976 (Hewitt et al., May 1, 2018, “Filamentary Devices for Treatment of Vascular Defects”) and 10939914 (Hewitt et al., Mar. 9, 2021, “Filamentary Devices for the Treatment of Vascular Defects”) disclose occlusion devices with permeable shells made of woven braided mesh having a variable mesh density and/or porosity.

U.S. patent applications 20170095254 (Hewitt et al., Apr. 6, 2017, “Filamentary Devices for Treatment of Vascular Defects”), 20190192166 (Hewitt et al., Jun. 27, 2019, “Filamentary Devices for Treatment of Vascular Defects”), and 20210106337 (Hewitt et al., Apr. 15, 2021, “Filamentary Devices for Treatment of Vascular Defects”) and U.S. Pat. No. 9,492,174 (Hewitt et al., Nov. 15, 2016, “Filamentary Devices for Treatment of Vascular Defects”), 10136896 (Hewitt et al., Nov. 27, 2018, “Filamentary devices for treatment of vascular defects”), and 10813645 (Hewitt et al., Oct. 27, 2020, “Filamentary Devices for Treatment of Vascular Defects”) disclose a self-expanding permeable shell having a radially constrained elongated state configured for delivery within a catheter lumen, an expanded state with a globular and longitudinally shortened configuration relative to the radially constrained state, and a plurality of elongate filaments that are woven together. U.S. patent application 20220192678 (Hewitt et al., Jun. 23, 2022, “Filamentary Devices for Treatment of Vascular Defects”) discloses an implant having a first permeable shell having a proximal hub and an open distal end and a second permeable shell having a distal hub and an open proximal end. U.S. patent application 20190223881 (Hewitt et al., Jul. 25, 2019, “Devices for Therapeutic Vascular Procedures”) discloses a self-expanding resilient permeable shell made from elongate resilient filaments with a distal region that extends beyond the distal end of the permeable shell.

U.S. patent application 20210007755 (Lorenzo et al., Jan. 14, 2021, “Intrasaccular Aneurysm Treatment Device with Varying Coatings”) discloses an aneurysm intrasaccular implant with coated regions. U.S. patent application 20190192168 (Lorenzo et al., Jun. 27, 2019, “Aneurysm Device and Delivery Method”) and U.S. patent Ser. No. 10/716,574 (Lorenzo et al., Jul. 21, 2020, “Aneurysm Device and Delivery Method”) disclose a self-expanding braid for treating an aneurysm, including a method for inverting and buckling a proximal segment. U.S. patent applications 20190223878 (Lorenzo et al., Jul. 25, 2019, “Aneurysm Device and Delivery System”) and 20200397447 (Lorenzo et al., Dec. 24, 2020, “Aneurysm Device and Delivery System”) and U.S. patent Ser. No. 10/905,430 (Lorenzo et al., Feb. 2, 2021, “Aneurysm Device and Delivery System”) disclose an expandable segment which radially expands inside an outer occlusive sack.

U.S. patent application 20200038034 (Maguire et al., Feb. 6, 2020, “Vessel Occluder”) discloses a vessel occluder with an expandable mesh portion having a flexible membrane that expands within a cavity of the expandable mesh portion. U.S. patent applications 20160249937 (Marchand et al., Sep. 1, 2016, “Multiple Layer Filamentary Devices for Treatment of Vascular Defects”) and application 20210282789 (Vu et al., Sep. 16, 2021, “Multiple Layer Devices for Treatment of Vascular Defects”) and U.S. Pat. No. 9,918,720 (Marchand et al., Mar. 20, 2018, “Multiple Layer Filamentary Devices for Treatment of Vascular Defects”) and 10238393 (Marchand et al., Mar. 26, 2019, “Multiple Layer Filamentary Devices for Treatment of Vascular Defects”) disclose devices and methods for treatment of a patient's vasculature, including a permeable shell and an inner structure configured to occlude blood flow therethrough.

U.S. patent application 20210346032 (Patterson et al., Nov. 11, 2021, “Devices for Treatment of Vascular Defects”) discloses an expandable stent for placement in a parent vessel proximal, near, or adjacent an aneurysm. U.S. patent application 20220211383 (Pereira et al., Jul. 7, 2022, “Systems and Methods for Treating Aneurysms”) discloses an apparatus for treating an aneurysm including an occlusion element coupled to an elongate delivery shaft and having a distal end, a proximal end, and a longitudinal axis extending between the distal end and the proximal end. U.S. patent applications 20170079662 (Rhee et al., Mar. 23, 2017, “Occlusive Devices”) and 20200038032 (Rhee et al., Feb. 6, 2020, “Occlusive Devices”) and U.S. patent Ser. No. 10/478,194 (Rhee et al., Nov. 19, 2019, “Occlusive Devices”) disclose an implant with a frame and a mesh component, wherein the mesh component has a first porosity and the frame has a second porosity.

U.S. patent applications 20140200607 (Sepetka et al., Jul. 17, 2014, “Occlusive Device”) and 20190274691 (Sepetka et al., Sep. 12, 2019, “Occlusive Device”) and U.S. patent Ser. No. 11/045,203 (Sepetka et al., Jun. 29, 2021, “Occlusive Device”) disclose multiple sequentially deployed occlusive devices that are connected together to create an extended length. U.S. patent applications 20170224350 (Shimizu et al., Aug. 10, 2017, “Devices for Vascular Occlusion”) and 20210228214 (Bowman et al., Jul. 29, 2021, “Devices for Vascular Occlusion”) and U.S. patent Ser. No. 10/729,447 (Shimizu et al., Aug. 4, 2020, “Devices for Vascular Occlusion”) and 10980545 (Bowman et al., Apr. 20, 2021, “Devices for vascular occlusion”) disclose an occlusive device, an occlusive device delivery system, method of using, method of delivering an occlusive device, and method of making an occlusive device to treat various intravascular conditions. U.S. patent application 20200375607 (Soto Del Valle et al., Dec. 3, 2020, “Aneurysm Device and Delivery System”) discloses a method of expanding mesh segments to form an outer occlusive sack and an inner occlusive sack.

U.S. patent application 20200367906 (Xu et al., Nov. 26, 2020, “Aneurysm Treatment with Pushable Ball Segment”) discloses a braided implant with a retractable dual proximal layer. U.S. patent applications 20200367893 (Xu et al., Nov. 26, 2020, “Layered Braided Aneurysm Treatment Device”), 20200367898 (Gorochow et al., Nov. 26, 2020, “Layered Braided Aneurysm Treatment Device”), and 20200367900 (Pedroso et al., Nov. 26, 2020, “Layered Braided Aneurysm Treatment Device With Corrugations”) and U.S. patent Ser. No. 10/653,425 (Gorochow et al., May 19, 2020, “Layered Braided Aneurysm Treatment Device”) and U.S. Pat. No. 11,413,046 (Gorochow et al., Aug. 16, 2022, “Layered Braided Aneurysm Treatment Device”) discloses a tubular braid comprising an open end, a pinched end, and a predetermined shape; wherein, in the predetermined shape, the tubular braid comprises: a first segment extending from the open end to a first inversion, a second segment encircled by the open end such that the second segment is only partially surrounded by the first segment and extending from the first inversion to a second inversion, and a third segment surrounded by the second segment and extending from the second inversion to the pinched end. U.S. patent application 20220087681 (Xu et al., Mar. 24, 2022, “Inverting Braided Aneurysm Implant with Dome Feature”) discloses an implant with a braid that extends across an aneurysm neck and anchors to the aneurysm's walls at least in the proximal portion of the aneurysm sac.

U.S. patent applications 20200367896 (Zaidat et al., Nov. 26, 2020, “Systems and Methods for Treating Aneurysms”) and 20220054141 (Zaidat et al., Feb. 24, 2022, “Systems and Methods for Treating Aneurysms”) and U.S. patent Ser. No. 11/202,636 (Zaidat et al., Dec. 21, 2021, “Systems and Methods for Treating Aneurysms”) disclose an apparatus for treating an aneurysm in a blood vessel with a first tubular mesh having a first end and a second end coupled together at a proximal end of the occlusion element.

### SUMMARY OF THE INVENTION

This invention is an intrasacular aneurysm occlusion device with two or more nested structures, including an inner embolic structure (e.g. inner mesh, net, braid, and/or stent) which is nested inside an outer embolic structure (e.g. outer mesh, net, braid, and/or stent). The device is inserted into and expanded within an aneurysm sac. In an example, inner and outer embolic structures of the device can be coaxial and/or concentric. In an example: an inner embolic structure can have an ellipsoidal, toroidal, inverted jug, or inverted bottle shape; and an outer embolic structure can have a spherical shape. In an example: an inner embolic structure can have an inverted jug, inverted bottle, or inverted teardrop shape; and an outer embolic structure can have a race-track-oval shape. In an example: an inner embolic structure can have a spherical, inverted jug, or inverted bottle shape; and an outer embolic structure can have a bowl, hemispherical, and/or paraboloidal shape. In an example, inner and outer embolic structures can be created separately and then attached to each other. Alternatively, inner and outer embolic structures can be formed from a common component (e.g. by radially-constraining and inverting a tubular mesh).

In an example, there can be an opening in an outer embolic structure through which embolic members and/or embolic material (e.g. embolic coils, hydrogels, microsponges, beads, ribbons, string-of-pearls strands, or congealing material) is inserted into the aneurysm sac. In an example, an outer embolic structure can be more flexible than an inner embolic structure. The less-flexible inner embolic structure can help to prevent the device from prolapsing out of the aneurysm sac and the more-flexible outer embolic structure can help the device to conform to the walls of an irregularly-shaped aneurysm sac, reducing the risk of recanalization. Insertion of embolic material into the flexible outer embolic structure can also help the flexible outer structure to better conform to the walls of an irregularly-shaped aneurysm. In an example, there can also be a valve which can be remotely closed by the operator of the device to close the opening after embolic members and/or material (e.g. embolic coils, hydrogels, microsponges, beads, ribbons, string-of-pearls strands, or congealing material) has been inserted. An intrasacular aneurysm occlusion device with such a nested structure can conform to the walls of an irregularly-shaped aneurysm and reduce blood flow through the neck of the aneurysm better than intrasacular alternatives such as embolic coils, embolic ribbons, or single-layer intrasacular aneurysm occlusion devices.

### BRIEF INTRODUCTION TO THE FIGURES

FIGS. 1 through 3 show an intrasacular aneurysm occlusion device with concentric inner and outer embolic structures.

FIG. 4 shows an intrasacular aneurysm occlusion device with a convex inner convex embolic structure, a convex outer embolic structure, and a valve through which embolic material is inserted between them.

FIG. 5 shows an intrasacular aneurysm occlusion device with spherical inner and outer embolic structures.

FIG. 6 shows an intrasacular aneurysm occlusion device with a spherical inner embolic structure, a spherical outer embolic structure, and an opening through which embolic material is inserted between them.

FIG. 7 shows an intrasacular aneurysm occlusion device with a bowl, hemispherical, and/or paraboloidal shaped inner embolic structure and a spherical outer embolic structure.

FIG. 8 shows an intrasacular aneurysm occlusion device with a bowl, hemispherical, and/or paraboloidal shaped inner embolic structure, a spherical outer embolic structure, and an opening through which embolic material is inserted into them.

FIG. 9 shows a mesh structure with a spherical and/or globular shape.

FIG. 10 shows a mesh structure with an ellipsoidal and/or oblate spheroid shape.

FIG. 11 shows a mesh structure with a barrel shape.

FIG. 12 shows a mesh structure with a canister shape.

FIG. 13 shows a mesh structure with a racetrack ovaloid shape.

FIG. 14 shows a mesh structure with a disk shape.

FIG. 15 shows a mesh structure with an apple shape.

FIG. 16 shows a mesh structure with a pumpkin shape.

FIG. 17 shows a mesh structure with a toroidal and/or doughnut shape.

FIG. 18 shows a mesh structure with a tree ornament (3D-revolved sine-wave) shape.

FIG. 19 shows a mesh structure with an egg shape.

FIG. 20 shows a mesh structure with a pear shape.

FIG. 21 shows a mesh structure with a teardrop shape.

FIG. 22 shows a mesh structure with a cardioid and/or heart shape.

FIG. 23 shows a mesh structure with a bullet shape.

FIG. 24 shows a mesh structure with a bowl, hemispherical, and/or paraboloidal shape.

FIG. 25 shows a mesh structure with a half-canister shape.

FIG. 26 shows a mesh structure with a funnel and/or half-hyperboloidal shape.

FIG. 27 shows a mesh structure with a jellyfish shape.

FIG. 28 shows a mesh structure with a 3D-revolved horse-shoe shape.

FIG. 29 shows a mesh structure with a jug and/or bottle shape.

FIG. 30 shows a mesh structure with an inverted jug and/or bottle shape.

FIG. 31 shows a mesh structure with a mushroom shape.

FIG. 32 shows a mesh structure with an hour-glass and/or hyperboloidal shape.

FIG. 33 shows a mesh structure with a peanut shape.

FIG. 34 shows a mesh structure with a dumbbell shape.

FIG. 35 shows a mesh structure with a reflected mushroom shape.

FIG. 36 shows a mesh structure with a Saturn shape.

FIG. 37 shows a mesh structure with a paper lantern shape.

FIG. 38 shows a mesh structure with a geodesic shape.

FIG. 39 shows an intrasacular aneurysm occlusion device with an inner embolic structure with a spherical shape and an outer embolic structure with a spherical shape.

FIG. 40 shows an intrasacular aneurysm occlusion device with an inner embolic structure with an ellipsoidal shape and an outer embolic structure with a spherical shape.

FIG. 41 shows an intrasacular aneurysm occlusion device with an inner embolic structure with a disk shape and an outer embolic structure with a spherical shape.

FIG. 42 shows an intrasacular aneurysm occlusion device with an inner embolic structure with a toroidal shape and an outer embolic structure with a spherical shape.

FIG. 43 shows an intrasacular aneurysm occlusion device with an inner embolic structure with a pear shape and an outer embolic structure with a spherical shape.

FIG. 44 shows an intrasacular aneurysm occlusion device with an inner embolic structure with a bowl, hemispherical, and/or paraboloidal shape and an outer embolic structure with a spherical shape.

FIG. 45 shows an intrasacular aneurysm occlusion device with an inner embolic structure with a funnel shape and an outer embolic structure with a spherical shape.

FIG. 46 shows an intrasacular aneurysm occlusion device with an inner embolic structure with an inverted jug shape and an outer embolic structure with a spherical shape.

FIG. 47 shows an intrasacular aneurysm occlusion device with an inner embolic structure with a toroidal shape and an outer embolic structure with an ellipsoidal shape.

FIG. 48 shows an intrasacular aneurysm occlusion device with an inner embolic structure with a cardioid shape and an outer embolic structure with an ellipsoidal shape.

FIG. 49 shows an intrasacular aneurysm occlusion device with an inner embolic structure with an inverted jug shape and an outer embolic structure with an ellipsoidal shape.

FIG. 50 shows an intrasacular aneurysm occlusion device with an inner embolic structure with a canister shape and an outer embolic structure with a canister shape.

FIG. 51 shows an intrasacular aneurysm occlusion device with an inner embolic structure with a racetrack ovaloid shape and an outer embolic structure with a canister shape.

FIG. 52 shows an intrasacular aneurysm occlusion device with an inner embolic structure with a tree ornament shape and an outer embolic structure with a canister shape.

FIG. 53 shows an intrasacular aneurysm occlusion device with an inner embolic structure with a teardrop shape and an outer embolic structure with a racetrack ovaloid shape.

FIG. 54 shows an intrasacular aneurysm occlusion device with an inner embolic structure with an inverted teardrop shape and an outer embolic structure with a racetrack ovaloid shape.

FIG. 55 shows an intrasacular aneurysm occlusion device with an inner embolic structure with an inverted jug shape and an outer embolic structure with a racetrack oval shape.

FIG. 56 shows an intrasacular aneurysm occlusion device with an inner embolic structure with an apple shape and an outer embolic structure with an apple shape.

FIG. 57 shows an intrasacular aneurysm occlusion device with an inner embolic structure with a pumpkin shape and an outer embolic structure with a pumpkin shape.

FIG. 58 shows an intrasacular aneurysm occlusion device with an inner embolic structure with an apple shape and an outer embolic structure with an egg shape.

FIG. 59 shows an intrasacular aneurysm occlusion device with an inner embolic structure with a cardioid shape and an outer embolic structure with an egg shape.

FIG. 60 shows an intrasacular aneurysm occlusion device with an inner embolic structure with a funnel shape and an outer embolic structure with a cardioid shape.

FIG. 61 shows an intrasacular aneurysm occlusion device with an inner embolic structure with an inverted teardrop shape and an outer embolic structure with a bullet shape.

FIG. 62 shows an intrasacular aneurysm occlusion device with an inner embolic structure with a spherical shape and an outer embolic structure with a bowl, hemispherical, and/or paraboloidal shape.

FIG. 63 shows an intrasacular aneurysm occlusion device with an inner embolic structure with an inverted bowl, hemispherical, and/or paraboloidal shape and an outer embolic structure with a bowl, hemispherical, and/or paraboloidal shape.

FIG. 64 shows an intrasacular aneurysm occlusion device with an inner embolic structure with an inverted jug shape and an outer embolic structure with a bowl, hemispherical, and/or paraboloidal shape.

FIG. 65 shows an intrasacular aneurysm occlusion device with an inner embolic structure with an inverted half-canister shape and an outer embolic structure with a half-canister shape.

FIG. 66 shows an intrasacular aneurysm occlusion device with an inner embolic structure with an inverted jug shape and an outer embolic structure with a half-canister shape.

### DETAILED DESCRIPTION OF THE FIGURES

In an example, an intrasacular aneurysm occlusion device with nested structures can comprise: a catheter that is configured to be inserted into a blood vessel; a resilient expandable member that is configured to travel through the catheter, be inserted into an aneurysm sac, and then be expanded within the aneurysm sac; and a flexible expandable member that is configured to travel through the catheter, be inserted into the aneurysm sack, and then be expanded within the aneurysm sac, wherein the resilient expandable member is inside the flexible expandable member. In an example, the resilient expandable member and/or the flexible expandable member can be selected from the group consisting of: a mesh, a net, a braid, and a stent. In an example, the device can also have an opening in the flexible expandable member through which embolic members and/or embolic material is inserted into the aneurysm sac. In an example, embolic members and/or embolic material can be embolic coils, hydrogels, microsponges, beads, ribbons, string-of-pearls strands, or congealing material. In an example, the device can also have a valve in the opening which can be remotely opened or closed by the operator of the device. In an example, embolic members and/or embolic material can fill space between the resilient expandable member and the flexible expandable member, but not space inside the resilient expandable member.

In an example, an intrasacular aneurysm occlusion device with nested structures can comprise: an inner convex mesh which is configured to be radially-expanded within an aneurysm; and an outer convex mesh which is configured to be radially-expanded within the aneurysm, wherein the inner convex mesh is inside the outer convex mesh. In an example, the device can include opening in the outer convex mesh through which embolic members and/or embolic material is inserted into the aneurysm sac. In an example, embolic members and/or embolic material can be embolic coils, hydrogels, microsponges, beads, ribbons, string-of-pearls strands, or congealing material. In an example, the device can include a valve in the opening which can be remotely opened or closed by the operator of the device. In an example, embolic members and/or embolic material can fill space between the inner convex mesh and the outer convex mesh, but not space inside the inner convex mesh.

In an example, an intrasacular aneurysm occlusion device with nested structures can comprise: an inner embolic structure; and an outer embolic structure, wherein the inner embolic structure is inside the outer embolic structure, and wherein the inner embolic structure and the outer embolic structure are configured to be inserted into and expanded within an aneurysm sac. In an example, an inner embolic structure and/or an outer embolic structure can be selected from the group consisting of: a mesh, a net, a braid, and a stent. In an example, the device can include an opening in the outer embolic structure through which embolic members and/or embolic material is inserted into the aneurysm sac. In an example, embolic members and/or embolic material can be embolic coils, hydrogels, microsponges, beads, ribbons, string-of-pearls strands, or congealing material. In an example, the device can include a valve in the opening which can be remotely opened or closed by the operator of the device. In an example, embolic members and/or embolic material can fill space between the inner embolic structure and the outer embolic structure, but not space inside the inner embolic structure. In an example, an inner embolic structure can have an ellipsoidal, toroidal, inverted jug, or inverted bottle shape; and an outer embolic structure can have a spherical shape. In an example, an inner embolic structure can have an inverted jug, inverted bottle, or inverted teardrop shape; and an outer embolic structure can have a race-track-oval shape. In an example, an inner embolic structure can have a spherical, inverted jug, or inverted bottle shape; and an outer embolic structure can have a bowl, hemispherical, and/or paraboloidal shape.

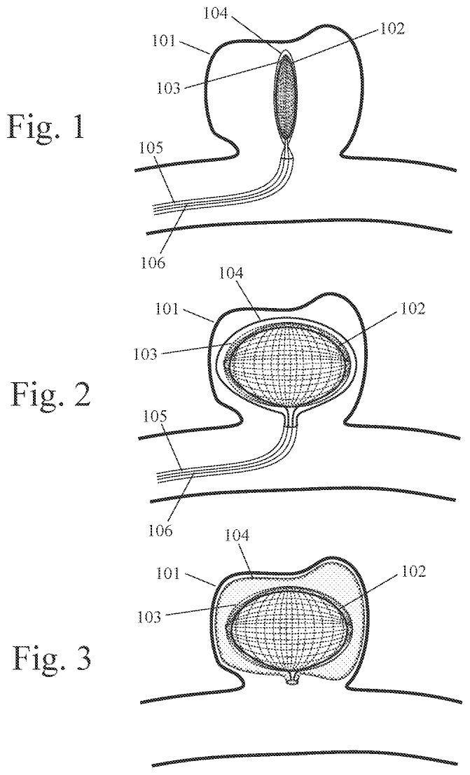

FIGS. 1 through 3 show an example of an intrasacular aneurysm occlusion device which can be described as using concentric resilient and non-resilient intrasacular members for aneurysm occlusion. More specifically, FIGS. 1 through 3 show an example of an intrasacular aneurysm occlusion device comprising: (a) a longitudinal catheter that is configured to be inserted into a blood vessel; (b) a resilient expandable member that is configured to travel through the longitudinal catheter, be inserted into an aneurysm sack, and then be expanded within the aneurysm sack; wherein this resilient expandable member resists contraction after it has been expanded; and wherein this resilient expandable member has a post-expansion shape that is selected from the group consisting of spherical, ellipsoidal, toroidal, compressed-sphere shaped, egg shaped, Saturn shaped, hour-glass shaped, peanut shaped, beehive shaped, and geodesic; and (c) a flexible expandable member that is configured to travel through the longitudinal catheter, be inserted into the aneurysm sack, and then be expanded within the aneurysm sack; wherein the resilient expandable member is inside the flexible expandable member; wherein the resilient expandable member is expanded before or while the flexible expandable member is expanded; and wherein the flexible expandable member is sufficiently flexible to substantively conform to the contours of the walls of the aneurysm sack when the flexible expandable member is expanded within the aneurysm.

FIGS. 1 through 3 also show an example of an intrasacular aneurysm occlusion device comprising: (a) a longitudinal catheter that is configured to be inserted into a blood vessel, wherein this blood vessel is the parent vessel from which an aneurysm has formed; (b) an expandable flexible member, wherein this expandable flexible member is configured to travel through the longitudinal catheter and to be inserted into the aneurysm sac; and wherein this flexible member is sufficiently flexible to substantially conform to the walls of an irregularly-shaped aneurysm sac after the flexible member has been expanded; and (c) an expandable resilient structure, wherein this expandable resilient structure is configured to travel through the longitudinal catheter and to be inserted into the aneurysm sac; wherein this structure is expanded inside the expandable flexible member; and wherein this structure resists compression after it has been expanded.

In an example, the expandable flexible member can be a net or mesh. In an example, the expandable resilient structure can be a stent. In an example, the expandable resilient structure can be substantially spherical or elliptical. In an example, the expandable resilient structure can be expanded before the expandable flexible member is expanded. In an example, the expandable resilient structure and the expandable flexible member can be expanded at substantially the same time. In an example, the total volume of an aneurysm sac can be X cubic units, wherein Y cubic units of the volume of the aneurysm would be filled by the largest-volume sphere that can be fitted into the aneurysm without stretching the aneurysm walls, wherein Z cubic units of the volume of the aneurysm can be filled by the expandable and flexible member; and wherein Z>[Y+0.5(X−Y)]. In an example, the total volume of an aneurysm sac can be X cubic units, wherein Y cubic units of the volume of the aneurysm would be filled by the largest-volume ellipsoid that can be fitted into the aneurysm without stretching the aneurysm walls, wherein Z cubic units of the volume of the aneurysm can be filled by the expandable flexible member; and wherein Z>[Y+0.5(X−Y)].

We now discuss the specific components of the example shown in FIGS. 1 through 3. FIGS. 1 through 3 show an example of an intrasacular aneurysm occlusion device comprising: an outer longitudinal catheter 105 that is configured to be inserted into a blood vessel from which aneurysm sac 101 has formed; an inner longitudinal catheter 106 within longitudinal catheter 105; an expandable flexible member 104 that is inserted and expanded within aneurysm sac 101, wherein flexible member 104 is sufficiently flexible to substantially conform to the walls of irregularly-shaped aneurysm sac 101; and an expandable resilient structure 103 that is expanded within flexible member 104 and resists compression after expansion.

In an example, expandable resilient structure 103 can be spherical or elliptical. In an example, expandable resilient structure 103 can be an expandable wire mesh or stent. In an example, expandable resilient structure 103 can be radially-expanded in plane which is substantially parallel to the plane that is defined by the central circumference of the aneurysm neck. In an example, resilient structure 103 can be expanded by inflation of a balloon 102 inside resilient structure 103. In an example, balloon 102 can be inflated by a fluid or gas that is delivered via catheter 105 or catheter 106. In an example, resilient structure 103 can self-expand after it exits catheter 105.

In an example, expandable flexible member 104 can be an expandable flexible net or mesh. In an example, flexible member 104 can be a porous fabric net or mesh. In an example, flexible member 104 can be a porous bag. In an example, expandable flexible member 104 can be a balloon with holes. In an example, flexible member 104 can be expanded by being filled with a plurality of embolic members. In an example, embolic members can be delivered into flexible member 104 through catheter 105 or catheter 106.

In an example, flexible member 104 can be compressed as it travels through a longitudinal catheter and then be expanded within aneurysm sac 101 after it is released from the catheter. In an example, flexible member 104 can be folded as it travels through a catheter and then be unfolded within aneurysm sac 101. In an example, flexible member 104 can be relatively loose or relaxed (in a lower-energy state) as it travels through a catheter and then be stretched or tense (in a higher-energy state) within aneurysm sac 101. In an example, flexible member 104 can be elastic or stretchable. In an example, flexible member 104 can be sufficiently elastic or stretchable that it expands when filled with an accumulation of embolic members, but not so elastic or stretchable that it allows embolic members to escape.

In an example, embolic members for filling flexible member 104 can be a plurality of soft, compressible members such as microsponges or blobs of gel. In an example, embolic members can be a plurality of hard, uncompressible members such as hard polymer spheres or beads. In an example, embolic members can be conveyed into flexible member 104 through catheter 105 or catheter 106. In an example, embolic members can be selected from the group consisting of: microsponges, pieces of gel, pieces of foam, beads, and embolic coils. In various examples, embolic members can be conveyed via a liquid flow, a moving belt, a wire loop, or an Archimedes screw.

In an example, this invention can comprise a method in which resilient structure 103 is expanded first and flexible member 104 is expanded second. In an example, this invention can comprise a method in which flexible member 104 is expanded first and resilient structure 103 is expanded second. In an example, this invention can comprise a method in which flexible member 104 and resilient structure 103 are expanded at substantially the same time.

As shown in FIGS. 1 through 3, an aneurysm sac can be irregular in shape. An aneurysm sac with an irregular shape will not be completely filled or spanned by a spherical or ellipsoid mass without stretching the aneurysm walls. In an example, the total volume of an aneurysm sac can be X cubic units (e.g. cubic millimeters). In an example, the maximum volume of the aneurysm which can be filled or spanned by a spherical or ellipsoid mass without stretching the aneurysm walls is Y cubic units (e.g. cubic millimeters). In an example, the total device shown in FIGS. 1 through 3 can fill more of the aneurysm than a spherical or ellipsoid mass because flexible member 104 is sufficiently flexible to fill or span the irregular perimeter of the aneurysm sac. This can have clinical benefits, such as reducing the chances of recanalization within the aneurysm sac. In an example, this device can fill or span more than 50% of the aneurysm volume which remains unfilled by a sphere or ellipsoid. In an example, this device can fill or span Z cubic units (e.g. cubic millimeters) of the volume of the aneurysm, wherein Z>[Y+0.5(X−Y)].

In an example, the combination of (a) an outer flexible member 104 that spans substantially the entire perimeter of the aneurysm sac 101 and (b) an inner resilient structure 103 can create a device that is sufficiently flexible to substantially fill the entire volume of an irregularly-shaped aneurysm sac, but also sufficiently resilient so as to compress against the aneurysm walls and not slip out of the aneurysm sac.

In an example, a flexible expanding member and an arcuate three-dimensional stent can be nested and/or concentric when they are in their second configurations, respectively. In this example, an aneurysm occlusion device has a single flexible expanding member and a single arcuate three-dimensional stent inside the flexible expanding member. In an example, an aneurysm occlusion device can have two nested arcuate three-dimensional stents within a single flexible expanding member. In an example, an aneurysm occlusion device can have two nested flexible expanding members around a single arcuate three-dimensional stent. In an example with two flexible expanding members, an outer flexible expanding member can have a first level of flexibility and an inner flexible expanding member can have a second level of flexibility, wherein the first level is greater than the second level. In this example, a flexible expanding member is filled and expanded by insertion of a plurality of embolic members.

In an example, an intrasacular aneurysm occlusion device can comprise: (a) a longitudinal catheter that is configured to be inserted into a blood vessel, wherein this blood vessel is the parent vessel from which an aneurysm has formed; (b) a flexible sac-filling portion of the device, wherein this flexible sac-filling portion is configured to travel through the longitudinal catheter and to be inserted into the aneurysm sac; and wherein this flexible sac-filling portion is sufficiently flexible to substantially conform to the walls of an irregularly-shaped aneurysm sac after the flexible sac-filling portion has been expanded; and (c) a resilient wider-than-neck portion of this device, wherein this resilient wider-than-neck portion of this device is configured to travel through the longitudinal catheter and to be inserted into the aneurysm sac; wherein this resilient wider-than-neck portion is expanded inside the flexible sac-filling portion; and wherein this resilient wider-than-neck portion resists compression after it has been expanded.

In an example, an intrasacular aneurysm occlusion device can comprise: (a) a longitudinal catheter that is configured to be inserted into a blood vessel; (b) a resilient expandable member that is configured to travel through the longitudinal catheter, be inserted into an aneurysm sack, and then be expanded within the aneurysm sack; and wherein this resilient expandable member resists contraction after it has been expanded; and (c) a flexible expandable member that is configured to travel through the longitudinal catheter, be inserted into the aneurysm sack, and then be expanded within the aneurysm sack; wherein the resilient expandable member is inside the flexible expandable member; wherein the resilient expandable member is expanded before or while the flexible expandable member is expanded; and wherein the flexible expandable member is sufficiently flexible to substantively conform to the contours of the walls of the aneurysm sack when the flexible expandable member is expanded within the aneurysm. In an example, the shape of the resilient expandable member can be selected from the group consisting of: apple shape; bowl shape; compressed-sphere shape; cylinder; disk; doughnut shape; egg shape; ellipsoid; Frisbee™ shape; frustum; hour-glass shape; oval; peanut shape; pear shape; pumpkin shape; ring shape; Saturn shape; sphere; tire shape; and torus.

In an example, an intrasacular aneurysm occlusion device can comprise: a resilient wider-than-neck portion with a first configuration as it is transported to an aneurysm sac and a second configuration after it has been expanded within the aneurysm sac; wherein the resilient wider-than-neck portion in its second configuration has a width which is larger than the diameter of the neck of the aneurysm sac; and wherein the resilient wider-than-neck in its second configuration has a first level of flexibility, elasticity, and/or malleability; and a flexible sac-filling portion with a first configuration as it is being transported to an aneurysm sac and a second configuration after it has been expanded within the aneurysm sac; wherein the flexible sac-filling portion is expanded from its first configuration to its second configuration by the insertion of embolic members into the flexible sac-filling portion; and wherein the flexible sac-filling portion in its second configuration has a second level of flexibility, elasticity, and/or malleability which is greater than the first level of flexibility, elasticity, and/or malleability. Other example variations discussed elsewhere in this disclosure or priority-linked disclosures can also be applied to this example where relevant.

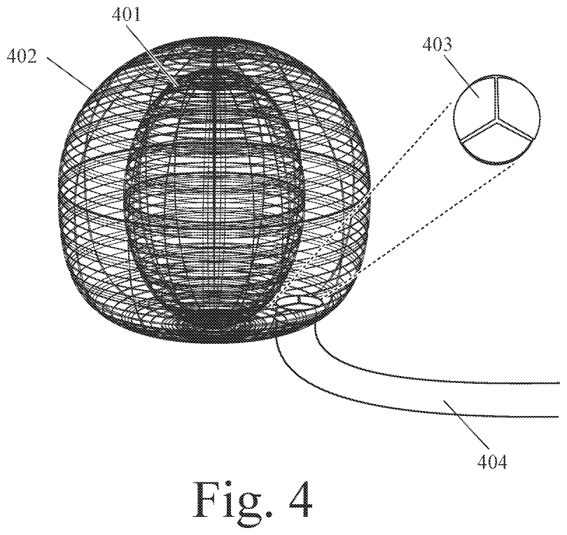

FIG. 4 shows an intrasacular aneurysm occlusion device comprising: an inner convex mesh 401 which is configured to be radially-expanded within an aneurysm; an outer convex mesh (or net) 402 which is configured to be radially-expanded within the aneurysm, wherein the inner convex mesh is inside the outer convex mesh; a valve 403 in the outer convex mesh (or net); and a catheter 404 through which embolic members (e.g. coils, hydrogels, microsponges, beads, or string-of-pearls strands) are inserted into the space between the inner convex mesh and the outer convex mesh. After the embolic members have been inserted, the valve is closed and the catheter is removed.

In an example, an inner convex mesh can be spherical. In an example, an inner convex mesh can be ellipsoidal. In an example, an inner convex mesh can be apple, barrel, or pear shaped. In an example, an inner convex mesh can be toroidal. In an example, an inner convex mesh can be hyperboloidal, dumbbell, peanut, or hour-glass shaped. In an example, an inner convex mesh can be disk shaped. In an example, an inner convex mesh can be shaped like a paper lantern. In an example, an inner convex mesh can be a wire mesh and/or frame. In an example, an inner convex mesh can be a woven or braided wire mesh and/or frame. In an example, an inner convex mesh can be made from metal and polymer components. In an example, an outer convex mesh (or net) can be spherical. In an example, an outer convex mesh can be ellipsoidal. In an example, an outer convex mesh can be apple, barrel, or pear shaped. In an example, an outer convex mesh can be shaped like a paper lantern. In an example, an outer convex mesh can be a wire mesh and/or frame. In an example, an outer convex mesh can be a woven or braided wire mesh and/or frame.

In an example, an outer convex mesh (or net) can be made from metal and polymer components. In an example, an inner convex mesh can be made from a metal and an outer convex mesh can be made from a polymer. In an example, inner and outer convex meshes can be nested. In an example, inner and outer convex meshes can be concentric. In an example, inner and outer convex meshes can be attached to each other. In an example, the proximal ends of inner and outer convex meshes can be attached to each other. In an example, the distal ends of inner and outer convex meshes can be attached to each other. In an example: the proximal ends of inner and outer convex meshes can be attached to each other; and the distal ends of inner and outer convex meshes can be attached to each other.

In an example, inner and outer convex meshes can both have the same durometer level. In an example, inner and outer convex meshes can both have the same elasticity. In an example, inner and outer convex meshes can both have the same porosity. In an example, an outer convex mesh (or net) can be less elastic than an inner convex mesh. In an example, an outer convex mesh can have a greater durometer level than an inner convex mesh. In an example, an outer convex mesh can have greater porosity than an inner convex mesh. In an example, an outer convex mesh can be more elastic than an inner convex mesh. In an example, an outer convex mesh can have a lower durometer level an inner convex mesh. In an example, an outer convex mesh can have lower porosity than an inner convex mesh.

In an example, a valve in an outer convex mesh (or net) can be off-center. In an example, a valve in an outer convex mesh (or net) can be offset from the central longitudinal axis of the mesh. In an example, there can be two or more off-center valves through a outer convex mesh. Alternatively, a valve in an outer convex mesh can be central to the cross-section of an outer convex mesh. In an example, a valve in an outer convex mesh can on the central longitudinal axis of the mesh. In an example, the cross-sectional area of a valve can be between 5% to 15% of the maximum cross-sectional area of an outer convex mesh. In an example, the cross-sectional area of a valve can be between 10% to 30% of the maximum cross-sectional area of an outer convex mesh. In an example, a valve can be a leaflet valve. In an example, a valve can be a bi-leaflet valve or tri-leaflet valve, analogous to a heart valve.

In an example, a valve can passively open when an embolic member is pushed through it and can passively close after the member passes through or when a portion of the member is detached. In an example, such a valve allows an embolic member to be inserted into an aneurysm, but the valve closes to reduce blood flow into the aneurysm after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug.

In an example, an outer convex mesh (or net) can be made from metal and polymer components. In an example, an inner convex mesh can be made from a metal and an outer convex mesh can be made from a polymer. In an example, inner and outer convex meshes can be nested. In an example, inner and outer convex meshes can be concentric. In an example, inner and outer convex meshes can be attached to each other. In an example, the proximal ends of inner and outer convex meshes can be attached to each other. In an example, the distal ends of inner and outer convex meshes can be attached to each other. In an example: the proximal ends of inner and outer convex meshes can be attached to each other; and the distal ends of inner and outer convex meshes can be attached to each other.

In an example, inner and outer convex meshes can both have the same durometer level. In an example, inner and outer convex meshes can both have the same elasticity. In an example, inner and outer convex meshes can both have the same porosity. In an example, an outer convex mesh (or net) can be less elastic than an inner convex mesh. In an example, an outer convex mesh can have a greater durometer level than an inner convex mesh. In an example, an outer convex mesh can have greater porosity than an inner convex mesh. In an example, an outer convex mesh can be more elastic than an inner convex mesh. In an example, an outer convex mesh can have a lower durometer level an inner convex mesh. In an example, an outer convex mesh can have lower porosity than an inner convex mesh.

In an example, a valve in an outer convex mesh (or net) can be off-center. In an example, a valve in an outer convex mesh (or net) can be offset from the central longitudinal axis of the mesh. In an example, there can be two or more off-center valves through a outer convex mesh. Alternatively, a valve in an outer convex mesh can be central to the cross-section of an outer convex mesh. In an example, a valve in an outer convex mesh can on the central longitudinal axis of the mesh. In an example, the cross-sectional area of a valve can be between 5% to 15% of the maximum cross-sectional area of an outer convex mesh. In an example, the cross-sectional area of a valve can be between 10% to 30% of the maximum cross-sectional area of an outer convex mesh. In an example, a valve can be a leaflet valve. In an example, a valve can be a bi-leaflet valve or tri-leaflet valve, analogous to a heart valve.

In an example, a valve can passively open when an embolic member is pushed through it and can passively close after the member passes through or when a portion of the member is detached. In an example, such a valve allows an embolic member to be inserted into an aneurysm, but the valve closes to reduce blood flow into the aneurysm after the embolic member has passed through the valve. In an example, an active valve can be remotely opened and/or closed by the operator of the device. In an example, an active valve can be remotely opened and/or closed by an operator by the application of electromagnetic energy. In an example, an active valve can be remotely opened and/or closed by an operator by pulling a filament. In an example, an active valve can be remotely opened and/or closed by an operator by pushing, pulling, or rotating a wire. In an example, an active valve can be remotely opened and/or closed by an operator by cutting, pulling, or pushing a flap or plug. Other example variations discussed elsewhere in this disclosure or priority-linked disclosures can also be applied to this example where relevant.

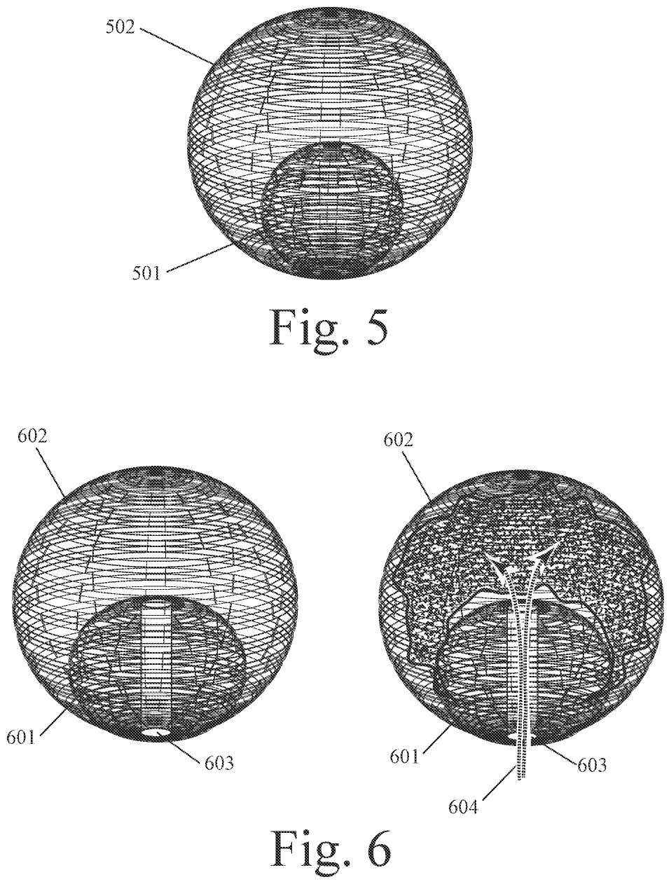

FIG. 5 shows an example of an intrasacular aneurysm occlusion device comprising: an inner embolic structure (e.g. inner mesh, net, or stent) 501; and an outer embolic structure (e.g. outer mesh, net, or stent) 502, wherein the inner embolic structure is inside the outer embolic structure, and wherein the inner embolic structure and the outer embolic structure are configured to be inserted and expanded within an aneurysm sac. In this example, both the inner embolic structure and the outer embolic structure are spherical.

In an example, the inner embolic structure and the outer embolic structure can be nested, with the inner embolic structure inside the hollow interior of the outer embolic structure. In an example, the inner embolic structure and the outer embolic structure can be concentric. In an example, the inner embolic structure and the outer embolic structure can be coaxial (e.g. sharing a common longitudinal axis).

In an example, an inner embolic structure (e.g. inner mesh, net, or stent) and an outer embolic structure (e.g. outer mesh, net, or stent) can be created as separate structures and then attached and/or connected to each other. In an example, the proximal (e.g. closer to an aneurysm neck) surface of an inner embolic structure can be attached to the proximal interior surface of the outer embolic structure. In an example, the distal (e.g. farther from an aneurysm neck) surface of an inner embolic structure can be attached to the distal interior surface of the outer embolic structure. In an example, the proximal (e.g. closer to an aneurysm neck) and distal (e.g. farther from the aneurysm neck) surfaces of an inner embolic structure can be attached to the proximal and distal interior surfaces, respectively, of the outer embolic structure.

In an example, an inner embolic structure (e.g. inner mesh, net, or stent) and an outer embolic structure (e.g. outer mesh, net, or stent) can be created as parts (e.g. portions, sections, undulations, bulbs, or loops) of the same continuous structure. In an example, an inner embolic structure and an outer embolic structure can be parts of the same mesh or net. In an example, an inner embolic structure and an outer embolic structure can be parts or sections of a radially-constrained tubular mesh or net. In an example, an inner embolic structure and an outer embolic structure can be parts or sections of a tubular mesh or net which is radially-constrained at multiple locations (e.g. at both ends and at least once in a mid-section). In an example, an inner embolic structure and an outer embolic structure can be parts or sections of a tubular structure which has been radially constrained at its ends. In an example, an inner embolic structure and an outer embolic structure can be parts or sections of a tubular structure which has been radially constrained at its middle and ends.

In an example, an inner embolic structure (e.g. inner mesh, net, or stent) can be connected to an outer embolic structure (e.g. outer mesh, net, or stent) by a toroidal ring or cylindrical band. In an example, an inner embolic structure (e.g. inner mesh, net, or stent) and an outer embolic structure (e.g. outer mesh, net, or stent) can be formed from a common continuous braided structure by a radially-constraining toroidal ring or cylindrical band. In an example, an inner embolic structure can be connected to an outer embolic structure by two toroidal rings or cylindrical bands, one inside the connection and one outside the connection.

In an example, another type of embolic members and/or material (e.g. coils, “string of pearls” embolic components, hydrogel particles, or a congealing substance) can be inserted into the interior of the inner embolic structure through the toroidal ring or cylindrical band. In an example, another type of embolic members and/or material (e.g. coils, “string of pearls” embolic components, hydrogel particles, or a congealing substance) can be inserted into the space between the inner embolic structure and the outer embolic structure through the toroidal ring or cylindrical band. In an example, another type of embolic members and/or material (e.g. coils, “string of pearls” embolic components, hydrogel particles, or a congealing substance) can be inserted into the both the interiors of inner embolic structure and the outer embolic structure through the toroidal ring or cylindrical band.

In an example, an inner embolic structure (e.g. inner mesh, net, or stent) can be attached and/or connected to an outer embolic structure (e.g. outer mesh, net, or stent) at one location and/or surface. In an example, an inner embolic structure can be attached and/or connected to an outer embolic structure at one central proximal location and/or surface, wherein proximal means closer to the neck of an aneurysm when the device is deployed in an aneurysm sac. In an example, an inner embolic structure can be attached and/or connected to an outer embolic structure at one central distal location and/or surface, wherein proximal means closer to the neck of an aneurysm when the device is deployed in an aneurysm sac.

In an example, an inner embolic structure can be attached and/or connected to an outer embolic structure at its most proximal location and/or surface, wherein proximal means closer to the neck of an aneurysm when the device is deployed in an aneurysm sac. In an example, an inner embolic structure can be attached and/or connected to an outer embolic structure at its most distal location and/or surface, wherein distal means farther from the neck of an aneurysm when the device is deployed in an aneurysm sac. In an example, an inner embolic structure (e.g. inner mesh, net, or stent) can be attached and/or connected to an outer embolic structure (e.g. outer mesh, net, or stent) at two location and/or surfaces: at its most proximal location and/or surface; and at its most distal location and/or surface.

In an example, an inner embolic structure (e.g. inner mesh, net, or stent) can have a spherical shape, ellipsoidal shape, oblate spheroid shape, racetrack ovaloid shape, and/or egg shape. In an example, an inner embolic structure can have a hemispherical shape, bowl shape, concave, and/or convex shape. In an example, an inner embolic structure can have a toroidal shape, doughnut shape, ring shape, cylindrical shape, and/or disk shape. In an example, an inner embolic structure can have an hour-glass shape, hyperboloidal shape, peanut shape, mushroom shape, dumbbell shape, and/or undulating shape. In an example, an inner embolic structure can have an apple shape, barrel shape, half-canister shape, pumpkin shape, pear shape, teardrop shape, tree ornament (revolved sine-wave), jug or bottle shape, cardioid shape, beehive, 3D revolved horse-shoe shape, paper lantern shape (e.g. pleated cylindrical shape), accordion shape (e.g. Weerdahl), and/or Saturn shape (e.g. spherical with one or more rings). In an example, an inner embolic structure can have a geodesic shape and/or frustal shape.

In an example, an inner embolic structure (e.g. inner mesh, net, or stent) and an outer embolic structure (e.g. outer mesh, net, or stent) can both have the same shape. In an example, an inner embolic structure and an outer embolic structure can have different shapes. In an example, an inner embolic structure and an outer embolic structure can have shapes with the same proximal-to-distal orientation. In an example, an inner embolic structure and an outer embolic structure can have shapes with different orientations. In an example, the shape of an inner embolic structure can be inverted from a proximal-to-distal orientation to a distal-to-proximal orientation. In an example, the shape of an outer embolic structure can be inverted from a proximal-to-distal orientation to a distal-to-proximal orientation.

In an example, an outer embolic structure can have a spherical shape, ellipsoidal shape, oblate spheroid shape, racetrack ovaloid shape, and/or egg shape. In an example, an outer embolic structure can have a hemispherical shape, bowl shape, concave, and/or convex shape. In an example, an outer embolic structure can have a toroidal shape, doughnut shape, ring shape, cylindrical shape, and/or disk shape. In an example, an outer embolic structure can have an hour-glass shape, hyperboloidal shape, peanut shape, mushroom shape, dumbbell shape, and/or undulating shape. In an example, an outer embolic structure can have an apple shape, barrel shape, half-canister shape, pumpkin shape, pear shape, teardrop shape, tree ornament (revolved sine-wave) (e.g. Christmas tree ornament) shape jug or bottle shape, cardioid shape, beehive, 3D revolved horse-shoe shape, paper lantern shape (e.g. pleated cylindrical shape), accordion shape (e.g. Weerdahl), and/or Saturn shape (e.g. spherical with one or more rings). In an example, an outer embolic structure can have a geodesic shape and/or frustal shape.

In an example, an inner embolic structure (e.g. inner mesh, net, or stent) can have a spherical shape and an outer embolic structure (e.g. outer mesh, net, or stent) can also have a spherical shape. In an example, an inner embolic structure can have a hemispherical shape and an outer embolic structure can have an ellipsoidal shape. In an example, an inner embolic structure can have a spherical shape and an outer embolic structure can have a toroidal shape. In an example, an inner embolic structure can have a hemispherical shape and an outer embolic structure can have a peanut shape. In an example, an inner embolic structure can have an hour-glass shape and an outer embolic structure can have an hour-glass shape. In an example, an inner embolic structure can have a bowl shape and an outer embolic structure can have an hour-glass shape. In an example, an inner embolic structure can have a toroidal shape and an outer embolic structure can have an ellipsoidal shape.

In an example, an inner embolic structure can have a cardioid shape and an outer embolic structure can have a half-canister shape. In an example, an inner embolic structure can have a tree ornament (revolved sine-wave) shape and an outer embolic structure can have a tree ornament (revolved sine-wave) shape. In an example, an inner embolic structure can have an hour-glass shape and an outer embolic structure can have a bowl shape. In an example, an inner embolic structure can have an apple shape and an outer embolic structure can have an ellipsoidal shape. In an example, an inner embolic structure can have a toroidal shape and an outer embolic structure can have a toroidal shape. In an example, an inner embolic structure can have a half-canister shape and an outer embolic structure can have an inverted half-barrel shape. In an example, an inner embolic structure can have a spherical shape and an outer embolic structure can have a hemispherical shape. In an example, an inner embolic structure can have a jug or bottle shape and an outer embolic structure can have a spherical shape. In an example, an inner embolic structure can have a peanut shape and an outer embolic structure can have a spherical shape.

In an example, an inner embolic structure can have an ellipsoidal shape and an outer embolic structure can have a hemispherical shape. In an example, an inner embolic structure can have a hemispherical shape and an outer embolic structure can have a toroidal shape. In an example, an inner embolic structure can have a spherical shape and an outer embolic structure can have an hour-glass shape. In an example, an inner embolic structure can have a hemispherical shape and an outer embolic structure can have a spherical shape. In an example, an inner embolic structure can have a spherical shape and an outer embolic structure can have an ellipsoidal shape.

In an example, an inner embolic structure can have a hemispherical shape and an outer embolic structure can have an apple shape. In an example, an inner embolic structure can have a spherical shape and an outer embolic structure can have a peanut shape. In an example, an inner embolic structure can have a bowl shape and an outer embolic structure can have a peanut shape. In an example, an inner embolic structure can have a pumpkin shape and an outer embolic structure can have a pumpkin shape. In an example, an inner embolic structure can have an hour-glass shape and an outer embolic structure can have an ellipsoidal shape. In an example, an inner embolic structure can have a hemispherical shape and an outer embolic structure can have a hemispherical shape.

In an example, an inner embolic structure can have a spherical shape and an outer embolic structure can have a spherical shape. In an example, an inner embolic structure can have a hemispherical shape and an outer embolic structure can have a bowl shape. In an example, an inner embolic structure can have a spherical shape and an outer embolic structure can have an apple shape. In an example, an inner embolic structure can have a jug or bottle and an outer embolic structure can have a hemispherical shape. In an example, an inner embolic structure can have a teardrop shape and an outer embolic structure can have a barrel shape. In an example, an inner embolic structure can have an apple shape and an outer embolic structure can have a toroidal shape.

In an example, an inner embolic structure can have an apple shape and an outer embolic structure can have a peanut shape. In an example, an inner embolic structure can have a cardioid shape and an outer embolic structure can have an egg or ovaloid shape. In an example, an inner embolic structure can have a racetrack ovaloid shape and an outer embolic structure can have a half-canister shape. In an example, an inner embolic structure can have an hour-glass shape and an outer embolic structure can have a peanut shape.

In an example, an inner embolic structure can have a cardioid shape and an outer embolic structure can have a pumpkin shape. In an example, an inner embolic structure can have a racetrack ovaloid shape and an outer embolic structure can have a half-canister shape. In an example, an inner embolic structure can have an hour-glass shape and an outer embolic structure can have a toroidal shape. In an example, an inner embolic structure can have a barrel shape and an outer embolic structure can have a barrel shape. In an example, an inner embolic structure can have a peanut shape and an outer embolic structure can have an ellipsoidal shape. In an example, an inner embolic structure can have an apple shape and an outer embolic structure can have an hour-glass shape.

In an example, an inner embolic structure can have a bowl shape and an outer embolic structure can have a toroidal shape. In an example, an inner embolic structure can have a peanut shape and an outer embolic structure can have an apple shape. In an example, an inner embolic structure can have an ellipsoidal shape and an outer embolic structure can have an ellipsoidal shape. In an example, an inner embolic structure can have a cardioid shape and an outer embolic structure can have a hemispherical shape. In an example, an inner embolic structure can have a spherical shape and an outer embolic structure can have a bowl shape. In an example, an inner embolic structure can have an hour-glass shape and an outer embolic structure can have a hemispherical shape.

In an example, an inner embolic structure can have a bowl shape and an outer embolic structure can have a spherical shape. In an example, an inner embolic structure can have a peanut shape and an outer embolic structure can have a toroidal shape. In an example, an inner embolic structure can have an ellipsoidal shape and an outer embolic structure can have a peanut shape. In an example, an inner embolic structure can have a bowl shape and an outer embolic structure can have an ellipsoidal shape. In an example, an inner embolic structure can have a peanut shape and an outer embolic structure can have a bowl shape. In an example, an inner embolic structure can have an ellipsoidal shape and an outer embolic structure can have a toroidal shape.

In an example, an inner embolic structure can have a toroidal shape and an outer embolic structure can have a spherical shape. In an example, an inner embolic structure can have a jug or bottle shape and an outer embolic structure can have a barrel shape. In an example, an inner embolic structure can have a peanut shape and an outer embolic structure can have a peanut shape. In an example, an inner embolic structure can have an apple shape and an outer embolic structure can have a bowl shape. In an example, an inner embolic structure can have a spherical shape and an outer embolic structure can have a carlavian curve shape. In an example, an inner embolic structure can have a toroidal shape and an outer embolic structure can have a peanut shape.

In an example, an inner embolic structure can have a bowl shape and an outer embolic structure can have an apple shape. In an example, an inner embolic structure can have a peanut shape and an outer embolic structure can have an hour-glass shape. In an example, an inner embolic structure can have an ellipsoidal shape and an outer embolic structure can have a bowl shape. In an example, an inner embolic structure can have a jug or bottle shape and an outer embolic structure can have a racetrack ovaloid shape. In an example, an inner embolic structure can have a teardrop shape and an outer embolic structure can have a pear shape.

In an example, an inner embolic structure can have a toroidal shape and an outer embolic structure can have an apple shape. In an example, an inner embolic structure can have an apple shape and an outer embolic structure can have a spherical shape. In an example, an inner embolic structure can have a toroidal shape and an outer embolic structure can have a bowl shape. In an example, an inner embolic structure can have an apple shape and an outer embolic structure can have an apple shape. In an example, an inner embolic structure can have a toroidal shape and an outer embolic structure can have an hour-glass shape. In an example, an inner embolic structure can have a jug or bottle shape and an outer embolic structure can have an racetrack ovaloid shape. In an example, an inner embolic structure can have an ellipsoidal shape and an outer embolic structure can have a spherical shape.

In an example, an inner embolic structure can have an apple shape and an outer embolic structure can have a hemispherical shape. In an example, an inner embolic structure can have a peanut shape and an outer embolic structure can have a hemispherical shape. In an example, an inner embolic structure can have an ellipsoidal shape and an outer embolic structure can have an apple shape. In an example, an inner embolic structure can have a toroidal shape and an outer embolic structure can have a hemispherical shape. In an example, an inner embolic structure can have a hemispherical shape and an outer embolic structure can have an hour-glass shape.

In an example, an inner embolic structure can have an hour-glass shape and an outer embolic structure can have a spherical shape. In an example, an inner embolic structure can have a bowl shape and an outer embolic structure can have a bowl shape. In an example, an inner embolic structure can have an hour-glass shape and an outer embolic structure can have an apple shape. In an example, an inner embolic structure can have a bowl shape and an outer embolic structure can have a hemispherical shape. In an example, an inner embolic structure can have an ellipsoidal shape and an outer embolic structure can have an hour-glass shape.

In an example, an inner embolic structure (e.g. inner mesh, net, or stent) can be folded or pleated. In an example, an inner embolic structure can have multiple folds or pleats. In an example, an inner embolic structure can have two or three folds or pleats. In an example, an inner embolic structure can have four or more folds or pleats. In an example, an inner embolic structure can have one or more proximal-to-distal (e.g. longitudinal) folds or pleats. In an example, an inner embolic structure can have one or more lateral folds or pleats. In an example, an inner embolic structure can have one or more circumferential folds or pleats. In an example, an inner embolic structure can be folded or pleated before it is expanded inside an aneurysm sac.

In an example, an inner embolic structure (e.g. inner mesh, net, or stent) can have lobes or undulations. In an example, an inner embolic structure can have multiple lobes or undulations. In an example, an inner embolic structure can have two or three lobes or undulations. In an example, an inner embolic structure can have four or more lobes or undulations. In an example, an inner embolic structure can have one or more proximal-to-distal (e.g. longitudinal) lobes or undulations. In an example, an inner embolic structure can have one or more lateral lobes or undulations. In an example, an inner embolic structure can have one or more circumferential lobes or undulations. In an example, an inner embolic structure can be lobed or undulating before it is expanded inside an aneurysm sac.

In an example, an outer embolic structure (e.g. outer mesh, net, or stent) can be folded or pleated. In an example, an outer embolic structure can have multiple folds or pleats. In an example, an outer embolic structure can have two or three folds or pleats. In an example, an outer embolic structure can have four or more folds or pleats. In an example, an outer embolic structure can have one or more proximal-to-distal (e.g. longitudinal) folds or pleats. In an example, an outer embolic structure can have one or more lateral folds or pleats. In an example, an outer embolic structure can have one or more circumferential folds or pleats. In an example, an outer embolic structure can be folded or pleated before it is expanded inside an aneurysm sac.

In an example, an outer embolic structure (e.g. outer mesh, net, or stent) can have lobes or undulations. In an example, an outer embolic structure can have multiple lobes or undulations. In an example, an outer embolic structure can have two or three lobes or undulations. In an example, an outer embolic structure can have four or more lobes or undulations. In an example, an outer embolic structure can have one or more proximal-to-distal (e.g. longitudinal) lobes or undulations. In an example, an outer embolic structure can have one or more lateral lobes or undulations. In an example, an outer embolic structure can have one or more circumferential lobes or undulations. In an example, an outer embolic structure can be lobed or undulating before it is expanded inside an aneurysm sac.

In an example, an intrasacular embolic structure (e.g. an inner structure and/or an outer structure) can have a shape selected from the group consisting of: spherical shape, ellipsoidal shape, oblate spheroid shape, barrel shape, canister shape, racetrack ovaloid shape, disk shape, apple shape, pumpkin shape, toroidal shape, doughnut shape, tree ornament (3D revolved sine-wave), egg shape, pear shape, teardrop shape, cardioid shape, bullet shape, hemispherical shape, bowl shape, half-canister shape, funnel shape, jellyfish shape, 3D revolved horse-shoe shape, jug or bottle shape, mushroom shape, hour-glass shape, hyperboloidal shape, peanut shape, dumbbell shape, reflected mushroom shape, Saturn shape, paper lantern shape, and geodesic shape.

In an example, there can be an opening an inner embolic structure (e.g. inner mesh, net, or stent) through which another type of embolic members and/or material (e.g. coils, “string of pearls” embolic components, congealing embolic material) can be inserted into the inner embolic structure. In an example, this opening can be centrally-located on the proximal surface of the inner embolic structure. In an example, this opening can be centrally-located on the aneurysm-neck-facing surface of the inner embolic structure. In an example, this opening can be the interior of a cylindrical band or tube which extends into the interior of the inner embolic structure.

In an example, an opening an inner embolic structure can be closed remotely by the operator of the device after embolic material has been inserted into the inner embolic structure. In an example, this opening can be closed remotely by the operator of the device by a closure mechanism selected from the group consisting of: activating a MEMS actuator, closing a leaflet valve, heating the opening, inserting a plug into the opening, melting the opening, operating an electromagnetic actuator, pulling a thread, suture, or string, pulling a wire, pulling catheter, pushing a wire, removing a strut which spans the opening, rotating a catheter, rotating a wire, shifting two layers the device relative to each other, tightening a loop around the opening, and transmitting electromagnetic energy.

In an example, there can be a lumen (through a tube, catheter, or cylindrical mesh) through the central proximal-to-distal axis of an inner embolic structure (e.g. inner mesh, net, or stent) through which another type of embolic members and/or material (e.g. coils, “string of pearls” embolic components, congealing embolic material) can be inserted into the space between the inner embolic structure and the outer embolic structure. In an example, there can be a lumen (through a tube, catheter, or cylindrical mesh) through the central proximal-to-distal axis of an inner embolic structure (e.g. inner mesh, net, or stent) through which another type of embolic members and/or material (e.g. coils, “string of pearls” embolic components, congealing embolic material) can be inserted into the space between the outer embolic structure and the walls of the aneurysm sac. In an example, this lumen can have a hyperboloidal and/or funnel shape.

In an example, an inner embolic structure can be connected to an outer embolic structure by two coaxial bands (e.g. rings, bands, tubes, and/or cylinders) comprising an inner band and outer band, wherein the inner and outer embolic structures are between the inner and outer bands. In an example, an inner embolic structure can be connected to an outer embolic structure by two coaxial bands (e.g. rings, bands, tubes, and/or cylinders) comprising an inner band and outer band, wherein the inner and outer embolic structures pass through the space between the inner and outer bands. In an example, an inner embolic structure and an outer embolic structure are radially-constrained by two coaxial bands (e.g. rings, bands, tubes, and/or cylinders) comprising an inner band and outer band, wherein the inner and outer embolic structures pass through the space between the inner and outer bands.

In an example, an inner embolic structure can be connected to an outer embolic structure by two coaxial bands (e.g. rings, bands, tubes, and/or cylinders) comprising an inner band and outer band, wherein the inner and outer embolic structures are between the inner and outer bands, and wherein other embolic structures or materials are inserted into the aneurysm sac through the center of the inner band. In an example, an inner embolic structure can be connected to an outer embolic structure by two coaxial bands (e.g. rings, bands, tubes, and/or cylinders) comprising an inner band and outer band, wherein the inner and outer embolic structures pass through the space between the inner and outer bands, and wherein other embolic structures or materials are inserted into the aneurysm sac through the center of the inner band. In an example, an inner embolic structure and an outer embolic structure are radially-constrained by two coaxial bands (e.g. rings, bands, tubes, and/or cylinders) comprising an inner band and outer band, wherein the inner and outer embolic structures pass through the space between the inner and outer bands, and wherein other embolic structures or materials are inserted into the aneurysm sac through the center of the inner band.

In an example, the size of an inner embolic structure (e.g. inner mesh, net, or stent) can be between 75% and 95% of the size of an outer embolic structure (e.g. outer mesh, net, or stent). In an example, the volume of an inner embolic structure (e.g. inner mesh, net, or stent) can be between 75% and 95% of the interior volume of an outer embolic structure (e.g. outer mesh, net, or stent). In an example, the size of an inner embolic structure can be between 45% and 80% of the size of an outer embolic structure. In an example, the volume of an inner embolic structure can be between 45% and 80% of the interior volume of an outer embolic structure.

In an example, the size of an inner embolic structure can be between 40% and 60% of the size of an outer embolic structure. In an example, the volume of an inner embolic structure can be between 40% and 60% of the interior volume of an outer embolic structure. In an example, the size of an inner embolic structure can be between 20% and 50% of the size of an outer embolic structure. In an example, the volume of an inner embolic structure can be between 20% and 50% of the interior volume of an outer embolic structure. In an example, an inner embolic structure (e.g. inner mesh, net, or stent) can be within the proximal half of the interior of an outer embolic structure (e.g. outer mesh, net, or stent). In an example, an inner embolic structure (e.g. inner mesh, net, or stent) can be within the proximal third of the interior of an outer embolic structure (e.g. outer mesh, net, or stent).

In an example, an inner embolic structure (e.g. inner mesh, net, or stent) and an outer embolic structure (e.g. outer mesh, net, or stent) can have different material and/or structural properties. In an example, an inner embolic structure can have a level of durometer which is greater than that of that of an outer embolic structure. In an example, an inner embolic structure (e.g. inner mesh, net, or stent) and an outer embolic structure (e.g. outer mesh, net, or stent) can have different levels of Young's Modulus. In an example, an inner embolic structure can have a level of porosity which is greater than that of that of an outer embolic structure.

In an example, an inner embolic structure can have a level of malleability which is greater than that of that of an outer embolic structure. In an example, an inner embolic structure can have a level of bendability which is greater than that of that of an outer embolic structure. In an example, an inner embolic structure and an outer embolic structure can have different levels of elasticity. In an example, an inner embolic structure can have a level of tensile strength which is less than that of that of an outer embolic structure. In an example, an inner embolic structure can have a level of durometer which is less than that of that of an outer embolic structure. In an example, an inner embolic structure can have a level of elasticity which is less than that of that of an outer embolic structure. In an example, an inner embolic structure can have a level of elasticity which is greater than that of that of an outer embolic structure. In an example, an inner embolic structure and an outer embolic structure can have different level of stiffness. In an example, an inner embolic structure can have a level of flexibility which is less than that of that of an outer embolic structure.

In an example, an inner embolic structure can have a level of flexibility which is greater than that of that of an outer embolic structure. In an example, an inner embolic structure can have a level of softness which is greater than that of that of an outer embolic structure. In an example, an inner embolic structure can have a level of softness which is less than that of that of an outer embolic structure. In an example, an inner embolic structure and an outer embolic structure can have different levels of tensile strength. In an example, an inner embolic structure and an outer embolic structure can have different thicknesses. In an example, an inner embolic structure and an outer embolic structure can have different levels of bendability. In an example, an inner embolic structure can have a level of stiffness which is less than that of that of an outer embolic structure. In an example, an inner embolic structure and an outer embolic structure can have different levels of flexibility. In an example, an inner embolic structure can have a level of tensile strength which is greater than that of that of an outer embolic structure.

In an example, an inner embolic structure can have a level of bendability which is less than that of that of an outer embolic structure. In an example, an inner embolic structure and an outer embolic structure can have different levels of malleability. In an example, an inner embolic structure can have a level of Young's Modulus which is less than that of that of an outer embolic structure. In an example, an inner embolic structure and an outer embolic structure can have different levels of softness.

In an example, an inner embolic structure can have a thickness which is less than that of that of an outer embolic structure. In an example, an inner embolic structure can have a level of porosity which is less than that of that of an outer embolic structure. In an example, an inner embolic structure and an outer embolic structure can have different levels of durometer. In an example, an inner embolic structure can have a level of stiffness which is greater than that of that of an outer embolic structure. In an example, an inner embolic structure and an outer embolic structure can have different levels of porosity. In an example, an inner embolic structure can have a level of Young's Modulus which is greater than that of that of an outer embolic structure. In an example, an inner embolic structure can have a level of malleability which is less than that of that of an outer embolic structure. In an example, an inner embolic structure can have a thickness which is greater than that of that of an outer embolic structure.

In an example, an intrasacular aneurysm occlusion device with inner and outer meshes can be made by the following steps: radially-constraining the proximal end of a tubular mesh; radially-constraining a mid-section of tubular mesh approximately one-third (e.g. between 25% and 45%) of the way from the proximal end to the distal end, thereby dividing the tube into proximal (approximately one-third) and distal (approximately two-thirds) portions; everting the distal portion over the proximal portion; and then radially-constraining the remaining open end of the tube. In an example, an intrasacular aneurysm occlusion device with inner and outer meshes can be made by the following steps: radially-constraining the proximal end of a tubular mesh; radially-constraining a mid-section of tubular mesh approximately one-fourth (e.g. between 20% and 30%) of the way from the proximal end to the distal end, thereby dividing the tube into proximal (approximately one-fourth) and distal (approximately three-fourths) portions; everting the distal portion over the proximal portion; and then radially-constraining the remaining open end of the tube.

In an example, an intrasacular aneurysm occlusion device with inner and outer meshes can be made by the following steps: radially-constraining the proximal end of a tubular mesh; radially-constraining a mid-section of tubular mesh approximately two-thirds (e.g. between 55% and 75%) of the way from the proximal end to the distal end, thereby dividing the tube into proximal (approximately two-thirds) and distal (approximately one-third) portions; everting the distal portion over the proximal portion; and then radially-constraining the remaining open end of the tube. In an example, an intrasacular aneurysm occlusion device with inner and outer meshes can be made by the following steps: radially-constraining the proximal end of a tubular mesh; radially-constraining a mid-section of tubular mesh approximately three-fourths (e.g. between 65% and 85%) of the way from the proximal end to the distal end, thereby dividing the tube into proximal (approximately three-fourths) and distal (approximately one-fourth) portions; everting the distal portion over the proximal portion; and then radially-constraining the remaining open end of the tube. Other example variations discussed elsewhere in this disclosure or priority-linked disclosures can also be applied to this example where relevant.

FIG. 6 shows two views, at two different times, of an example of an intrasacular aneurysm occlusion device comprising: an inner embolic structure (e.g. an inner mesh, net, or stent) 601; an outer embolic structure (e.g. an outer mesh, net, or stent) 602, wherein the inner embolic structure is inside the outer embolic structure, and wherein the inner embolic structure and the outer embolic structure are configured to be inserted and expanded within an aneurysm sac; an opening 603 through the inner embolic structure; and embolic material (e.g. coils, embolic particles, “string-of-pearls” embolic components, or congealing material) 604 which is inserted through the opening into the space between the inner embolic structure and the outer embolic structure. The left side of FIG. 6 shows this device at a first point in time, before embolic material has been inserted into the device. The right side of FIG. 6 shows this device at a second point in time, as embolic material is being inserted into the device. In this example, both the inner embolic structure and the outer embolic structure are spherical. In this example, the axes of the inner embolic structure and the outer embolic structure are aligned. Other example variations discussed elsewhere in this disclosure or priority-linked disclosures can also be applied to this example where relevant.

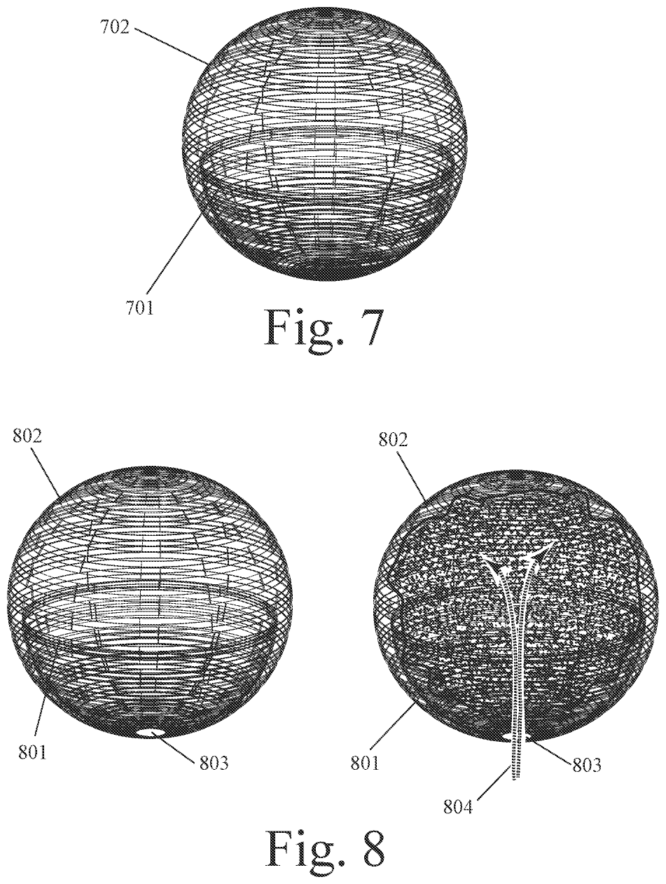

FIG. 7 shows an example of an intrasacular aneurysm occlusion device comprising: an inner embolic structure (e.g. inner mesh, net, or stent) 701; and an outer embolic structure (e.g. outer mesh, net, or stent) 702, wherein the inner embolic structure is inside the outer embolic structure, and wherein the inner embolic structure and the outer embolic structure are configured to be inserted and expanded within an aneurysm sac. In this example, the inner embolic structure is bowl-shaped (and/or hemispherical) and the outer embolic structure is spherical. In this example, the axes of the inner embolic structure and the outer embolic structure are aligned. Other example variations discussed elsewhere in this disclosure or priority-linked disclosures can also be applied to this example where relevant.

FIG. 8 shows two views, at two different times, of an example of an intrasacular aneurysm occlusion device comprising: an inner embolic structure (e.g. an inner mesh, net, or stent) 801; an outer embolic structure (e.g. an outer mesh, net, or stent) 802, wherein the inner embolic structure is inside the outer embolic structure, and wherein the inner embolic structure and the outer embolic structure are configured to be inserted and expanded within an aneurysm sac; an opening 803 through the inner embolic structure; and embolic material (e.g. coils, embolic particles, “string-of-pearls” embolic components, or congealing material) 804 which is inserted through the opening into the interior of the device. The left side of FIG. 8 shows this device at a first point in time, before embolic material has been inserted into the device. The right side of FIG. 8 shows this device at a second point in time, as embolic material is being inserted into the device. In this example, the inner embolic structure is bowl-shaped (and/or hemispherical) and the outer embolic structure is spherical. In this example, the axes of the inner embolic structure and the outer embolic structure are aligned. Other example variations discussed elsewhere in this disclosure or priority-linked disclosures can also be applied to this example where relevant.

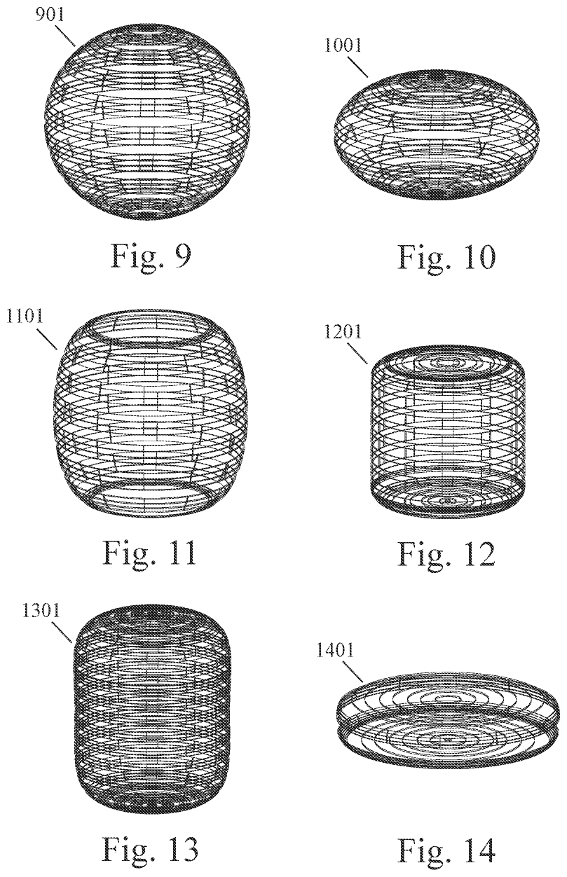

FIG. 9 shows an example of a mesh structure 901 with a spherical shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a spherical shape.

FIG. 10 shows an example of a mesh structure 1001 with an ellipsoidal and/or oblate spheroid shape. An ellipsoid is a three-dimensional surface of which all cross sections are either ellipses or circles. An oblate spheroid is an example of an ellipsoid created by compressing a sphere along an axis. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a ellipsoidal or oblate spheroid shape.

FIG. 11 shows an example of a mesh structure 1101 with a barrel shape. A barrel shape can be described as a generally-cylindrical prism whose end circumferences are smaller than its mid-section circumference. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a barrel shape.

FIG. 12 shows an example of a mesh structure 1201 with a canister shape. An canister shape can be described as a generally-cylindrical prism, wherein edge perimeter edges of its lower and upper surfaces are rounded. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a canister shape.

FIG. 13 shows an example of a mesh structure 1301 with a racetrack ovaloid shape. A racetrack ovaloid can be described is a generally-oval shape. wherein at least half of the lengths of its longitudinal sides are straight and parallel to each other. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a racetrack ovaloid shape.

FIG. 14 shows an example of a mesh structure 1401 with a disk shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a disk shape.

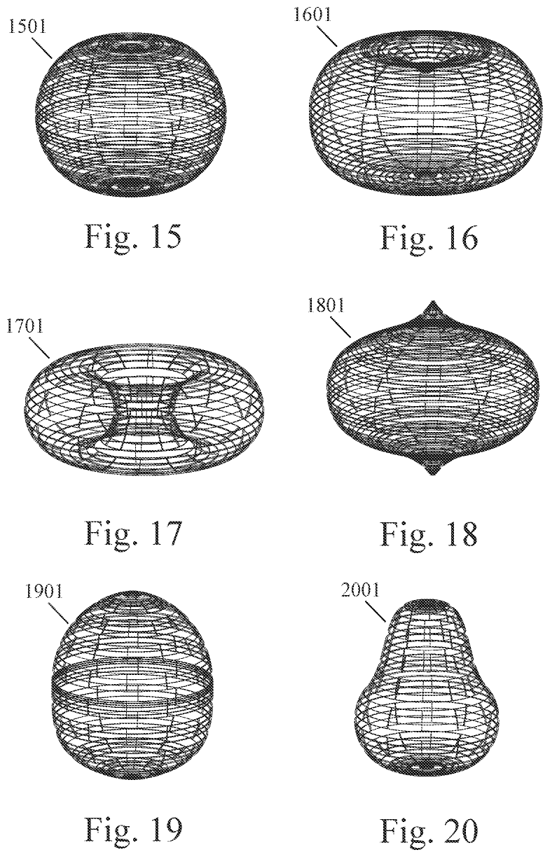

FIG. 15 shows an example of a mesh structure 1501 with a apple shape. An apple shape can be described as sphere or oblate spheroid with a concave (e.g. funnel-shaped) indentation which is centrally located on its upper surface and on its lower surface. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a apple shape.

FIG. 16 shows an example of a mesh structure 1601 with a pumpkin shape. A pumpkin shape is similar to an apple shape except that the concave indentations of a pumpkin shape are larger and its ratio of width to height is larger than that of an apple shape. A pumpkin shape can also be described as a surface of revolution which is formed by revolving between 60% and 80% of a circle in three dimensions around an axis that is coplanar with the circle. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a pumpkin shape.

FIG. 17 shows an example of a mesh structure 1701 with a toroidal and/or doughnut shape. A torus is a surface of revolution which is formed by revolving a circle in three dimensions around an axis that is coplanar with the circle. A doughnut is a torus-shaped baked item preferred by Homer Simpson which is made out of Doh! In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a toroidal or doughnut shape.

FIG. 18 shows an example of a mesh structure 1801 with a tree ornament (3D-revolved sine-wave) shape. This tree ornament shape can be described as an ellipsoid with both an upward-facing spire and a downward-facing spire. This tree ornament shape can also be described as a 3D-revolution of a single phase of a sine wave. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a tree ornament (3D-revolved sine-wave) shape.

FIG. 19 shows an example of a mesh structure 1901 with an egg shape. In another example, the inner embolic structure and/or the outer embolic structure of a multi-part (e.g. multi-layer) intrasacular aneurysm occlusion device can have such a egg shape.

FIG. 20 shows an example of a mesh structure 2001 with a pear shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a pear shape.

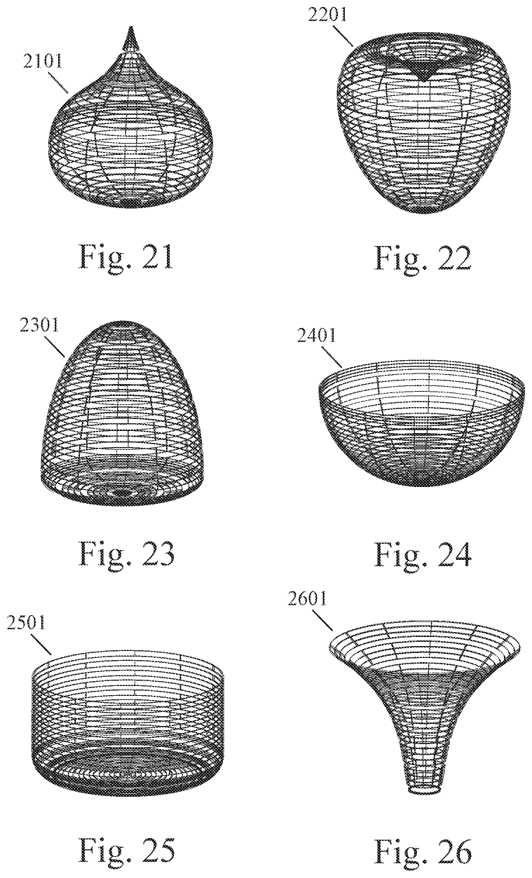

FIG. 21 shows an example of a mesh structure 2101 with a teardrop shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a teardrop shape.

FIG. 22 shows an example of a mesh structure 2201 with a cardioid and/or heart shape. More specifically, this mesh structure is the 3D revolution of a cardioid or heart shape. A cardioid is a curve traced by a point on the perimeter of a circle that is rolled around a fixed circle of the same radius. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a cardioid shape.

FIG. 23 shows an example of a mesh structure 2301 with a bullet shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a bullet shape.

FIG. 24 shows an example of a mesh structure 2401 with a bowl, hemispherical, and/or paraboloidal shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a bowl, hemispherical, and/or paraboloidal shape.

FIG. 25 shows an example of a mesh structure 2501 with a half-canister shape. A canister shape can be described as a generally-cylindrical, wherein edge perimeter edges of its lower and upper surfaces are rounded. This half-canister shape is created by cutting the canister in half laterally. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a half-canister shape.

FIG. 26 shows an example of a mesh structure 2601 with a funnel and/or half-hyperboloidal shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a funnel shape.

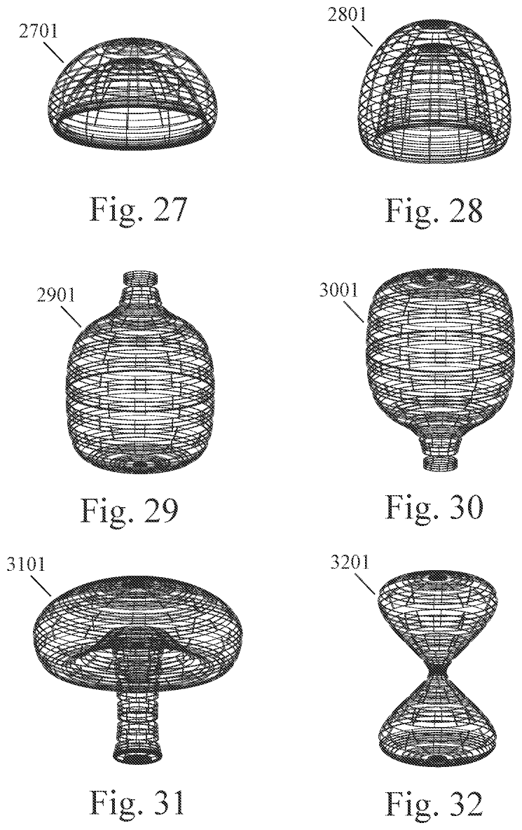

FIG. 27 shows an example of a mesh structure 2701 with a jellyfish shape. The jellyfish shape discussed herein can be formed by inverting one side of a hollow sphere or ellipsoid mesh into the other side of the sphere or ellipsoid mesh, but stopping the inversion action before the originally-polar ends of the sphere or ellipsoid contact each other. In an example, the post-inversion distance between the originally-polar ends can be between 5% and 35% of the pre-invention distance between them. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a jellyfish shape.

FIG. 28 shows an example of a mesh structure 2801 with a 3D-revolved horse-shoe shape. This shape can be created by revolving a horse-shoe shape in three dimensions around the central longitudinal axis of the horse shoe. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a 3D-revolved horse-shoe shape.

FIG. 29 shows an example of a mesh structure 2901 with a jug and/or bottle shape. A jug and/or bottle shape can be defined as a shape with a generally-cylindrical lower portion and a funnel-shaped upper portion, wherein the maximum diameter of the upper portion is the diameter of the top of the lower portion. Alternatively, a jug and/or bottle shape can be defined as a shape with a generally-cylindrical main body and an upwardly-tapered neck above the main body. Also, a diameter-to-neck ratio can be defined as: the maximum diameter of the cylindrical lower portion divided by the minimum diameter of the neck portion. If one wishes to distinguish between a bottle shape and a jug shape, a jug shape can be defined as having a diameter-to-neck ratio of 5 or greater and a bottle shape can be defined as a having a diameter-to-neck ratio between 2 and 5. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a jug or bottle shape.

FIG. 30 shows an example of a mesh structure 3001 with an inverted jug and/or bottle shape which is a vertically-inverted version of the jug and/or bottle shape which was defined in the narrative accompanying FIG. 29. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such an inverted jug or bottle shape.

FIG. 31 shows an example of a mesh structure 3101 with a mushroom shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a mushroom shape.

FIG. 32 shows an example of a mesh structure 3201 with an hour-glass and/or hyperboloidal shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a hour-glass or hyperboloidal shape.

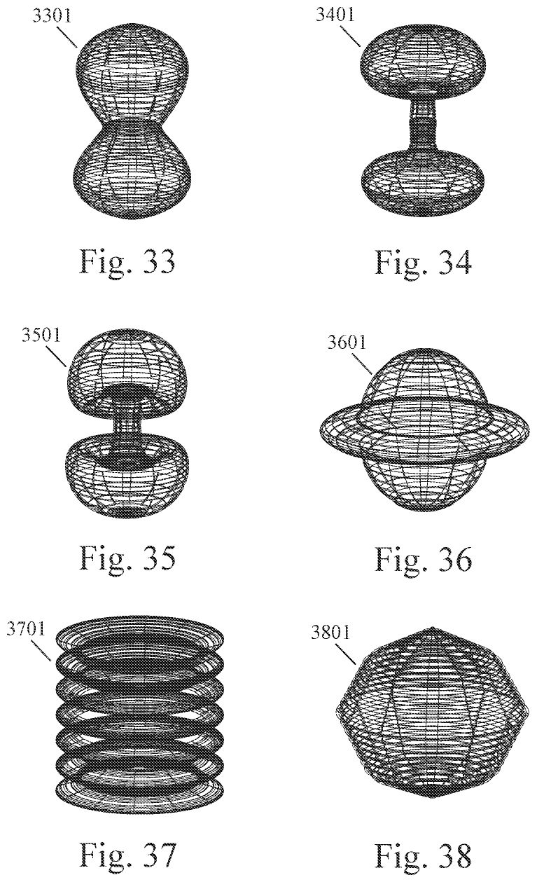

FIG. 33 shows an example of a mesh structure 3301 with a peanut shape. To further clarify what is meant by a peanut shape, a peanut shape can be described as globular lobes which are connected by a narrower arcuate mid-section. A peanut shape can also be described the 3D-revolution of two parallel and overlapping, but not perfectly aligned, lemniscate figures. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a peanut shape.

FIG. 34 shows an example of a mesh structure 3401 with a dumbbell shape. A dumbbell shape can be described as two globular lobes or disks which are connected by a narrow column. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a dumbbell shape.

FIG. 35 shows an example of a mesh structure 3501 with a reflected mushroom shape. This “reflected mushroom” or “double mushroom” shape is created by combining first and second mushroom shapes, wherein the second mushroom is a reflection (around a base horizontal line) of the first mushroom. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a reflected mushroom shape.

FIG. 36 shows an example of a mesh structure 3601 with a Saturn shape. A Saturn shape can be described as sphere or ellipsoid with a ring and/or torus shaped protrusion around its central circumference. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a Saturn shape.

FIG. 37 shows an example of a mesh structure 3701 with a paper lantern shape. A paper lantern shape can be described as a barrel or canister shape with undulating (e.g. sinusoidal, zigzag, and/or pleated) longitudinal sides. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a paper lantern shape.

FIG. 38 shows an example of a mesh structure 3801 with a geodesic shape. In an example, a mesh (e.g. net, mesh, or stent) with this shape can be an intrasacular aneurysm occlusion device by itself. In another example, the inner embolic structure and/or the outer embolic structure of a multi-part (e.g. multi-layer) intrasacular aneurysm occlusion device can have such a geodesic shape.

In an example, an intrasacular aneurysm occlusion device can comprise: an inner embolic structure (e.g. net, mesh, or stent) with a funnel shape; and an outer embolic structure (e.g. net, mesh, or stent) with a cardioid shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with a teardrop shape; and an outer embolic structure (e.g. net, mesh, or stent) with a racetrack ovaloid shape, wherein the inner embolic structure is (at least partially) inside the outer embolic structure, and wherein the inner embolic structure and the outer embolic structure are configured to be inserted into and expanded within an aneurysm sac.

In another example, an intrasacular aneurysm occlusion device can comprise: an inner embolic structure (e.g. net, mesh, or stent) with a racetrack ovaloid shape; and an outer embolic structure (e.g. net, mesh, or stent) with a canister shape, wherein the inner embolic structure is (at least partially) inside the outer embolic structure, and wherein the inner embolic structure and the outer embolic structure are configured to be inserted into and expanded within an aneurysm sac.

Alternatively, an intrasacular aneurysm occlusion device can comprise: an inner embolic structure (e.g. net, mesh, or stent) with a pear shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with an inverted bowl, hemispherical, and/or paraboloidal shape; and an outer embolic structure (e.g. net, mesh, or stent) with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In another example, an intrasacular aneurysm occlusion device can comprise: an inner embolic structure (e.g. net, mesh, or stent) with a toroidal shape; and an outer embolic structure (e.g. net, mesh, or stent) with an ellipsoidal shape, wherein the inner embolic structure is (at least partially) inside the outer embolic structure, and wherein the inner embolic structure and the outer embolic structure are configured to be inserted into and expanded within an aneurysm sac.

In an example, an intrasacular aneurysm occlusion device can comprise: an inner embolic structure (e.g. net, mesh, or stent) with a bowl, hemispherical, and/or paraboloidal shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with an inverted half-canister shape; and an outer embolic structure (e.g. net, mesh, or stent) with a half-canister shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with a tree ornament shape; and an outer embolic structure (e.g. net, mesh, or stent) with a canister shape, wherein the inner embolic structure is (at least partially) inside the outer embolic structure, and wherein the inner embolic structure and the outer embolic structure are configured to be inserted into and expanded within an aneurysm sac.

In another example, an intrasacular aneurysm occlusion device can comprise: an inner embolic structure (e.g. net, mesh, or stent) with an inverted jug and/or bottle shape; and an outer embolic structure (e.g. net, mesh, or stent) with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In another example, an intrasacular aneurysm occlusion device can comprise: an inner embolic structure (e.g. net, mesh, or stent) with an inverted jug and/or bottle shape; and an outer embolic structure (e.g. net, mesh, or stent) with an ellipsoidal shape, wherein the inner embolic structure is (at least partially) inside the outer embolic structure, and wherein the inner embolic structure and the outer embolic structure are configured to be inserted into and expanded within an aneurysm sac.

Alternatively, an intrasacular aneurysm occlusion device can comprise: an inner embolic structure (e.g. net, mesh, or stent) with an ellipsoidal shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac.

In another example, an intrasacular aneurysm occlusion device can comprise: an inner embolic structure (e.g. net, mesh, or stent) with an inverted jug and/or bottle shape; and an outer embolic structure (e.g. net, mesh, or stent) with a half-canister shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with an inverted jug and/or bottle shape; and an outer embolic structure (e.g. net, mesh, or stent) with a racetrack ovaloid shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with an inverted jug and/or bottle shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with a spherical shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac.

In another example, an intrasacular aneurysm occlusion device can comprise: an inner embolic structure (e.g. net, mesh, or stent) with a spherical shape; and an outer embolic structure (e.g. net, mesh, or stent) with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. Alternatively, an intrasacular aneurysm occlusion device can comprise: an inner embolic structure (e.g. net, mesh, or stent) with an apple shape; and an outer embolic structure (e.g. net, mesh, or stent) with an apple shape, wherein the inner embolic structure is (at least partially) inside the outer embolic structure, and wherein the inner embolic structure and the outer embolic structure are configured to be inserted into and expanded within an aneurysm sac.

In an example, an intrasacular aneurysm occlusion device can comprise: an inner embolic structure (e.g. net, mesh, or stent) with a canister shape; and an outer embolic structure (e.g. net, mesh, or stent) with a canister shape, wherein the inner embolic structure is (at least partially) inside the outer embolic structure, and wherein the inner embolic structure and the outer embolic structure are configured to be inserted into and expanded within an aneurysm sac. In another example, an intrasacular aneurysm occlusion device can comprise: an inner embolic structure (e.g. net, mesh, or stent) with a funnel shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac.

In an example, an intrasacular aneurysm occlusion device can comprise: an inner embolic structure (e.g. net, mesh, or stent) with a toriodal shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac. In another example, an intrasacular aneurysm occlusion device can comprise: an inner embolic structure (e.g. net, mesh, or stent) with a cardioid shape; and an outer embolic structure (e.g. net, mesh, or stent) with an egg shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with a disk shape; and an outer embolic structure (e.g. net, mesh, or stent) with a spherical shape, wherein the inner embolic structure is (at least partially) inside the outer embolic structure, and wherein the inner embolic structure and the outer embolic structure are configured to be inserted into and expanded within an aneurysm sac.

In an example, an intrasacular aneurysm occlusion device can comprise: an inner embolic structure (e.g. net, mesh, or stent) with a pumpkin shape; and an outer embolic structure (e.g. net, mesh, or stent) with a pumpkin shape, wherein the inner embolic structure is (at least partially) inside the outer embolic structure, and wherein the inner embolic structure and the outer embolic structure are configured to be inserted into and expanded within an aneurysm sac. In an example, an intrasacular aneurysm occlusion device can comprise: an inner embolic structure (e.g. net, mesh, or stent) with an apple shape; and an outer embolic structure (e.g. net, mesh, or stent) with an egg shape, wherein the inner embolic structure is (at least partially) inside the outer embolic structure, and wherein the inner embolic structure and the outer embolic structure are configured to be inserted into and expanded within an aneurysm sac. In another example, an intrasacular aneurysm occlusion device can comprise: an inner embolic structure (e.g. net, mesh, or stent) with an inverted teardrop shape; and an outer embolic structure (e.g. net, mesh, or stent) with a racetrack ovaloid shape, wherein the inner embolic structure is (at least partially) inside the outer embolic structure, and wherein the inner embolic structure and the outer embolic structure are configured to be inserted into and expanded within an aneurysm sac.

Alternatively, an intrasacular aneurysm occlusion device can comprise: an inner embolic structure (e.g. net, mesh, or stent) with a cardioid shape; and an outer embolic structure (e.g. net, mesh, or stent) with an ellipsoidal shape, wherein the inner embolic structure is (at least partially) inside the outer embolic structure, and wherein the inner embolic structure and the outer embolic structure are configured to be inserted into and expanded within an aneurysm sac. In another example, an intrasacular aneurysm occlusion device can comprise: an inner embolic structure (e.g. net, mesh, or stent) with an inverted teardrop shape; and an outer embolic structure (e.g. net, mesh, or stent) with a bullet shape, wherein the inner embolic structure is (at least partially) inside the outer embolic structure, and wherein the inner embolic structure and the outer embolic structure are configured to be inserted into and expanded within an aneurysm sac.

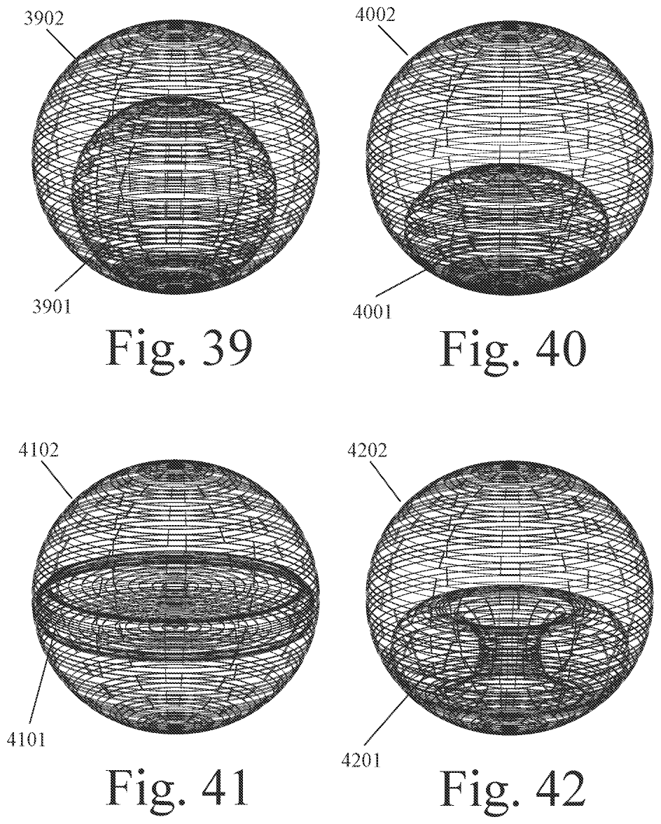

FIG. 39 shows an example of an intrasacular aneurysm occlusion device comprising: an inner embolic structure (e.g. mesh, net, braid, and/or stent) 3901 with a spherical shape; and an outer embolic structure (e.g. mesh, net, braid, and/or stent) 3902 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 40 shows an example of an intrasacular aneurysm occlusion device comprising: an inner embolic structure (e.g. mesh, net, braid, and/or stent) 4001 with an ellipsoidal shape; and an outer embolic structure (e.g. mesh, net, braid, and/or stent) 4002 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 41 shows an example of an intrasacular aneurysm occlusion device comprising: an inner embolic structure (e.g. mesh, net, braid, and/or stent) 4101 with a disk shape; and an outer embolic structure (e.g. mesh, net, braid, and/or stent) 4102 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 42 shows an example of an intrasacular aneurysm occlusion device comprising: an inner embolic structure (e.g. mesh, net, braid, and/or stent) 4201 with a toroidal shape; and an outer embolic structure (e.g. mesh, net, braid, and/or stent) 4202 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

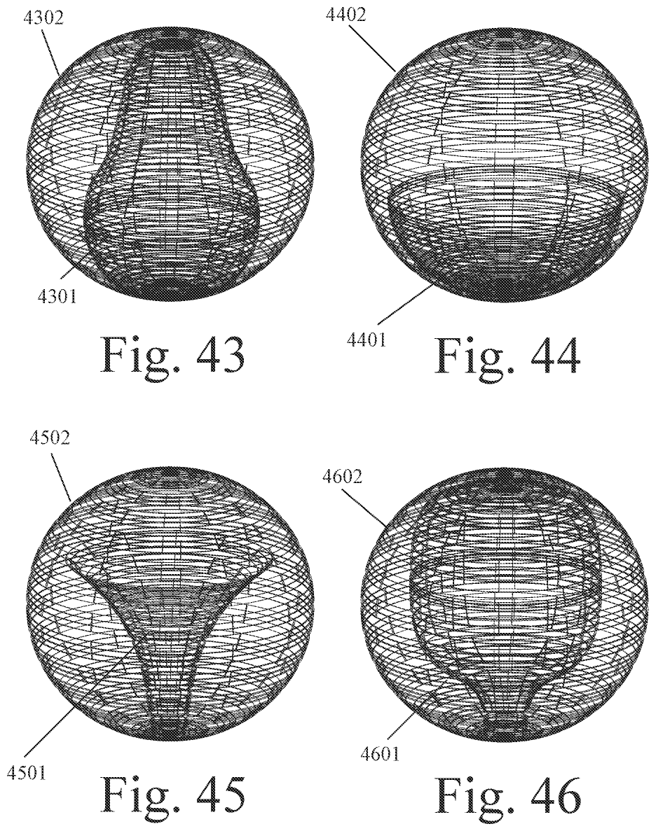

FIG. 43 shows an example of an intrasacular aneurysm occlusion device comprising: an inner embolic structure (e.g. mesh, net, braid, and/or stent) 4301 with a pear shape; and an outer embolic structure (e.g. mesh, net, braid, and/or stent) 4302 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 44 shows an example of an intrasacular aneurysm occlusion device comprising: an inner embolic structure (e.g. mesh, net, braid, and/or stent) 4401 with a bowl, hemispherical, and/or paraboloidal shape; and an outer embolic structure (e.g. mesh, net, braid, and/or stent) 4402 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 45 shows an example of an intrasacular aneurysm occlusion device comprising: an inner embolic structure (e.g. mesh, net, braid, and/or stent) 4501 with a funnel shape; and an outer embolic structure (e.g. mesh, net, braid, and/or stent) 4502 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 46 shows an example of an intrasacular aneurysm occlusion device comprising: an inner embolic structure (e.g. mesh, net, braid, and/or stent) 4601 with an inverted jug and/or bottle shape; and an outer embolic structure (e.g. mesh, net, braid, and/or stent) 4602 with a spherical shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

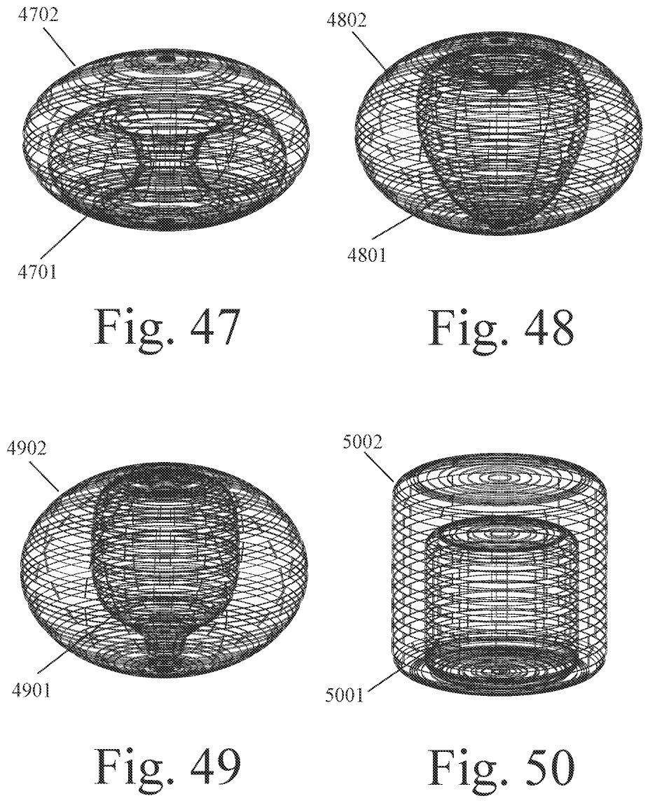

FIG. 47 shows an example of an intrasacular aneurysm occlusion device comprising: an inner embolic structure (e.g. mesh, net, braid, and/or stent) 4701 with a toroidal shape; and an outer embolic structure (e.g. mesh, net, braid, and/or stent) 4702 with an ellipsoidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 48 shows an example of an intrasacular aneurysm occlusion device comprising: an inner embolic structure (e.g. mesh, net, braid, and/or stent) 4801 with a cardioid shape; and an outer embolic structure (e.g. mesh, net, braid, and/or stent) 4802 with an ellipsoidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 49 shows an example of an intrasacular aneurysm occlusion device comprising: an inner embolic structure (e.g. mesh, net, braid, and/or stent) 4901 with an inverted jug and/or bottle shape; and an outer embolic structure (e.g. mesh, net, braid, and/or stent) 4902 with an ellipsoidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 50 shows an example of an intrasacular aneurysm occlusion device comprising: an inner embolic structure (e.g. mesh, net, braid, and/or stent) 5001 with a canister shape; and an outer embolic structure (e.g. mesh, net, braid, and/or stent) 5002 with a canister shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

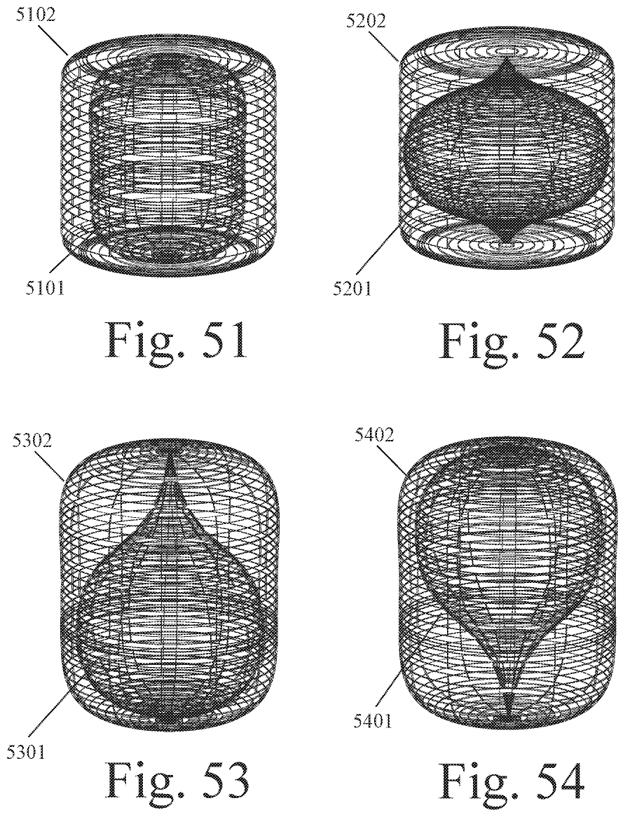

FIG. 51 shows an example of an intrasacular aneurysm occlusion device comprising: an inner embolic structure (e.g. mesh, net, braid, and/or stent) 5101 with a racetrack ovaloid shape; and an outer embolic structure (e.g. mesh, net, braid, and/or stent) 5102 with a canister shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 52 shows an example of an intrasacular aneurysm occlusion device comprising: an inner embolic structure (e.g. mesh, net, braid, and/or stent) 5201 with a tree ornament shape; and an outer embolic structure (e.g. mesh, net, braid, and/or stent) 5202 with a canister shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 53 shows an example of an intrasacular aneurysm occlusion device comprising: an inner embolic structure (e.g. mesh, net, braid, and/or stent) 5301 with a teardrop shape; and an outer embolic structure (e.g. mesh, net, braid, and/or stent) 5302 with a racetrack ovaloid shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 54 shows an example of an intrasacular aneurysm occlusion device comprising: an inner embolic structure (e.g. mesh, net, braid, and/or stent) 5401 with an inverted teardrop shape; and an outer embolic structure (e.g. mesh, net, braid, and/or stent) 5402 with a racetrack ovaloid shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

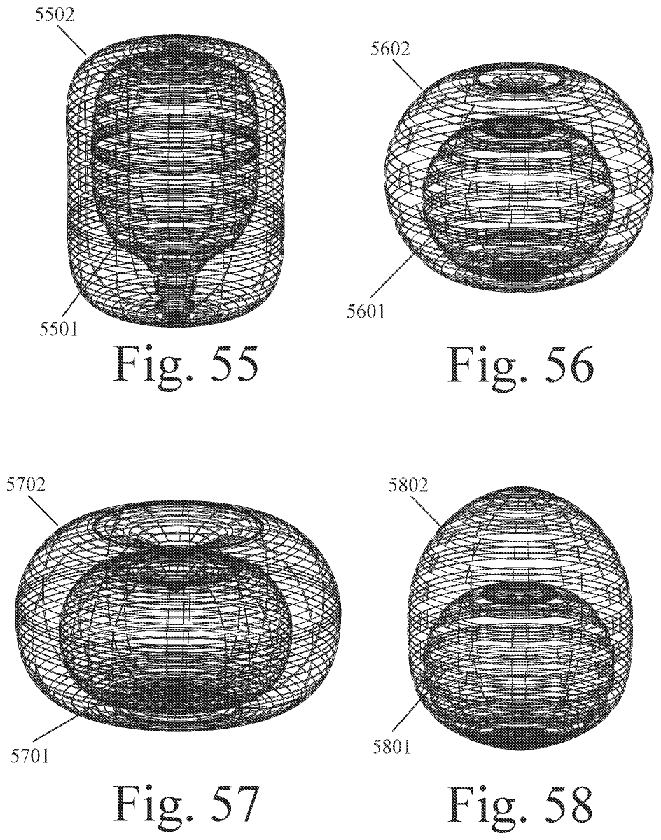

FIG. 55 shows an example of an intrasacular aneurysm occlusion device comprising: an inner embolic structure (e.g. mesh, net, braid, and/or stent) 5501 with an inverted jug and/or bottle shape; and an outer embolic structure (e.g. mesh, net, braid, and/or stent) 5502 with a racetrack oval shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 56 shows an example of an intrasacular aneurysm occlusion device comprising: an inner embolic structure (e.g. mesh, net, braid, and/or stent) 5601 with an apple shape; and an outer embolic structure (e.g. mesh, net, braid, and/or stent) 5602 with an apple shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 57 shows an example of an intrasacular aneurysm occlusion device comprising: an inner embolic structure (e.g. mesh, net, braid, and/or stent) 5701 with a pumpkin shape; and an outer embolic structure (e.g. mesh, net, braid, and/or stent) 5702 with a pumpkin shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 58 shows an example of an intrasacular aneurysm occlusion device comprising: an inner embolic structure (e.g. mesh, net, braid, and/or stent) 5801 with an apple shape; and an outer embolic structure (e.g. mesh, net, braid, and/or stent) 5802 with an egg shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

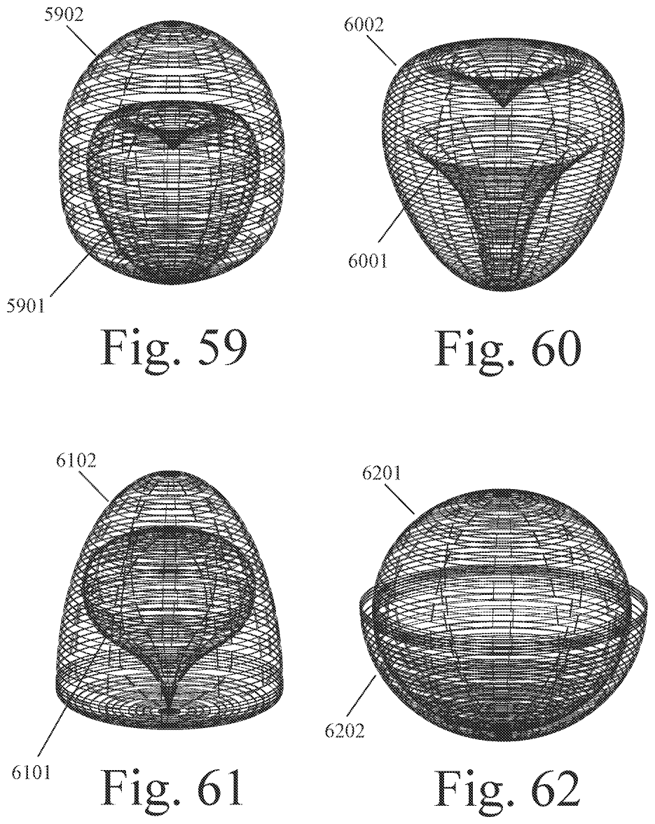

FIG. 59 shows an example of an intrasacular aneurysm occlusion device comprising: an inner embolic structure (e.g. mesh, net, braid, and/or stent) 5901 with a cardioid shape; and an outer embolic structure (e.g. mesh, net, braid, and/or stent) 5902 with an egg shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 60 shows an example of an intrasacular aneurysm occlusion device comprising: an inner embolic structure (e.g. mesh, net, braid, and/or stent) 6001 with a funnel shape; and an outer embolic structure (e.g. mesh, net, braid, and/or stent) 6002 with a cardioid shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 61 shows an example of an intrasacular aneurysm occlusion device comprising: an inner embolic structure (e.g. mesh, net, braid, and/or stent) 6101 with an inverted teardrop shape; and an outer embolic structure (e.g. mesh, net, braid, and/or stent) 6102 with a bullet shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 62 shows an example of an intrasacular aneurysm occlusion device comprising: an inner embolic structure (e.g. mesh, net, braid, and/or stent) 6201 with a spherical shape; and an outer embolic structure (e.g. mesh, net, braid, and/or stent) 6202 with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

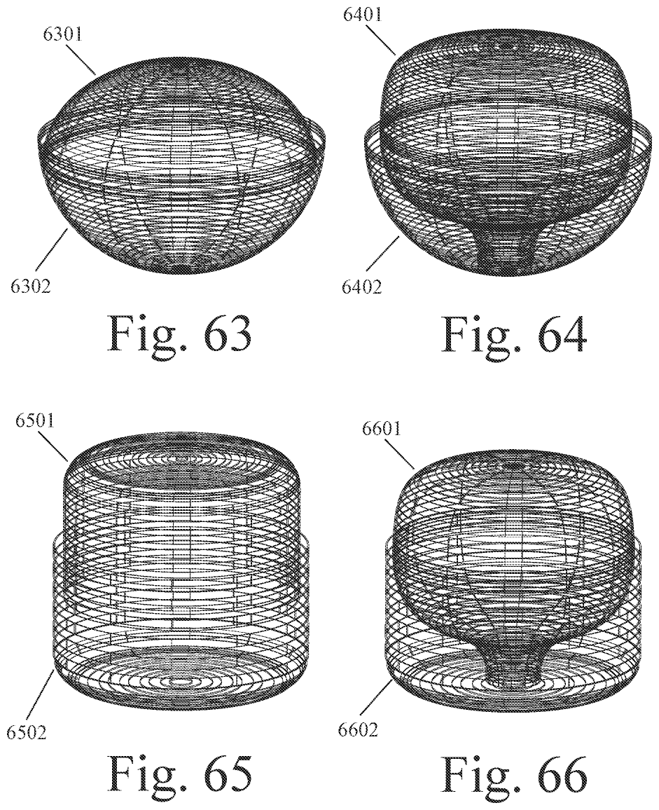

FIG. 63 shows an example of an intrasacular aneurysm occlusion device comprising: an inner embolic structure (e.g. mesh, net, braid, and/or stent) 6301 with an inverted bowl, hemispherical, and/or paraboloidal shape; and an outer embolic structure (e.g. mesh, net, braid, and/or stent) 6302 with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 64 shows an example of an intrasacular aneurysm occlusion device comprising: an inner embolic structure (e.g. mesh, net, braid, and/or stent) 6401 with an inverted jug and/or bottle shape; and an outer embolic structure (e.g. mesh, net, braid, and/or stent) 6402 with a bowl, hemispherical, and/or paraboloidal shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 65 shows an example of an intrasacular aneurysm occlusion device comprising: an inner embolic structure (e.g. mesh, net, braid, and/or stent) 6501 with an inverted half-canister shape; and an outer embolic structure (e.g. mesh, net, braid, and/or stent) 6502 with a half-canister shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

FIG. 66 shows an example of an intrasacular aneurysm occlusion device comprising: an inner embolic structure (e.g. mesh, net, braid, and/or stent) 6601 with an inverted jug and/or bottle shape; and an outer embolic structure (e.g. mesh, net, braid, and/or stent) 6602 with a half-canister shape, wherein the inner embolic structure is (at least partially) inside the outer embolic structure, and wherein the inner embolic structure and the outer embolic structure are configured to be inserted into and expanded within an aneurysm sac. In an example, the inner embolic structure and the outer embolic structure can be separately-created components which are attached together. In an example, the inner embolic structure and outer embolic structure can be two portions which are formed from a continuous component (e.g. a continuous tubular mesh or braid which is radially-constrained). In an example, this device can further comprise a (closeable) opening through the inner embolic structure and/or the outer embolic structure through which embolic members or material (e.g. coils, hydrogels, other embolic particles, string-of-pearls embolic strands, or congealing material) is inserted into the aneurysm sac. Example variations discussed elsewhere in this disclosure or in priority-linked disclosures can be applied to this example where relevant.

I claim:

1. An intrasacular aneurysm occlusion device with nested structures comprising:
a catheter that is configured to be inserted into a blood vessel;
a resilient expandable member that is configured to travel through the catheter, be inserted into an aneurysm sac, and then be expanded within the aneurysm sac; and
a flexible expandable member that is configured to travel through the catheter, be inserted into the aneurysm sack, and then be expanded within the aneurysm sac, wherein the resilient expandable member is inside the flexible expandable member.

2. The device in claim 1 wherein the resilient expandable member and/or the flexible expandable member is selected from the group consisting of: a mesh, a net, a braid, and a stent.

3. The device in claim 1 which further comprises an opening in the flexible expandable member through which embolic members and/or embolic material is inserted into the aneurysm sac.

4. The device in claim 3 wherein embolic members and/or embolic material comprises embolic coils, hydrogels, microsponges, beads, ribbons, string-of-pearls strands, or congealing material.

5. The device in claim 3 which further comprises a valve in the opening which can be remotely opened or closed by the operator of the device.

6. The device in claim 3 wherein embolic members and/or embolic material fills space between the resilient expandable member and the flexible expandable member, but not space inside the resilient expandable member.

7. An intrasacular aneurysm occlusion device with nested structures comprising:
an inner convex mesh which is configured to be radially-expanded within an aneurysm; and
an outer convex mesh which is configured to be radially-expanded within the aneurysm, wherein the inner convex mesh is inside the outer convex mesh.

8. The device in claim 7 which further comprises an opening in the outer convex mesh through which embolic members and/or embolic material is inserted into the aneurysm sac.

9. The device in claim 8 wherein embolic members and/or embolic material comprises embolic coils, hydrogels, microsponges, beads, ribbons, string-of-pearls strands, or congealing material.

10. The device in claim 8 which further comprises a valve in the opening which can be remotely opened or closed by the operator of the device.

11. The device in claim 8 wherein embolic members and/or embolic material fills space between the inner convex mesh and the outer convex mesh, but not space inside the inner convex mesh.

12. An intrasacular aneurysm occlusion device with nested structures comprising:
an inner embolic structure; and
an outer embolic structure, wherein the inner embolic structure is inside the outer embolic structure, and wherein the inner embolic structure and the outer embolic structure are configured to be inserted into and expanded within an aneurysm sac.

13. The device in claim 12 wherein the inner embolic structure and/or the outer embolic structure is selected from the group consisting of: a mesh, a net, a braid, and a stent.

14. The device in claim 12 which further comprises an opening in the outer embolic structure through which embolic members and/or embolic material is inserted into the aneurysm sac.

15. The device in claim 14 wherein embolic members and/or embolic material comprises embolic coils, hydrogels, microsponges, beads, ribbons, string-of-pearls strands, or congealing material.

16. The device in claim 14 which further comprises a valve in the opening which can be remotely opened or closed by the operator of the device.

17. The device in claim 14 wherein embolic members and/or embolic material fills space between the inner embolic structure and the outer embolic structure, but not space inside the inner embolic structure.

18. The device in claim 12 wherein: the inner embolic structure has an ellipsoidal, toroidal, inverted jug, or inverted bottle shape; and the outer embolic structure has a spherical shape.

19. The device in claim 12 wherein: the inner embolic structure has an inverted jug, inverted bottle, or inverted teardrop shape; and the outer embolic structure has a race-track-oval shape.

20. The device in claim 12 wherein: the inner embolic structure has a spherical, inverted jug, or inverted bottle shape; and the outer embolic structure has a bowl, hemispherical, and/or paraboloidal shape.
